(19) United States
(12) Patent Application Publication
(10) Pub. No.: US 2010/0061984 A1
(43) Pub. Date: Mar. 11, 2010

(54) COMPOSITIONS AND METHODS FOR MODULATION OF SUPPRESSOR T CELL ACTIVATION

(75) Inventors: Mark L. Greene, Penn Valley, PA (US); Sandra W. Saouaf, Moorestown, NJ (US); Bin Li, Chesterbrook, PA (US); Hongtao Zhang, Paoli, PA (US); Wayne Hancock, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: The Trustees of the University of Pennsylvania, Philadelphia, PA (US); The Children's Hospital of Philadelphia, Philadelphia, PA (US)

(21) Appl. No.: 12/161,192

(22) PCT Filed: Jan. 22, 2007
(86) PCT No.: PCT/US07/01677

§ 371 Date: Jan. 21, 2009

Related U.S. Application Data
(60) Provisional application No. 60/760,549, filed on Jan. 20, 2006.
(60) Provisional application No. 60/794,670, filed on Apr. 24, 2006.

(51) Int. Cl. A61K 39/395; A61K 39/00; A61K 35/14; A61K 38/12; A61K 48/00; A61K 31/675; A61K 31/519; A61K 31/52; A61K 31/4355; A61K 31/436

(52) U.S. Cl. 424/133.1; 424/184.1; 424/93.7; 514/8; 514/44 R; 514/90; 514/249; 514/263.2; 514/263.31; 514/291

(57) ABSTRACT

Methods of treating autoimmune disorders, coronary artery disease, allergy symptoms, allograft rejection sepsis/toxic shock are disclosed. Some methods comprise administering one or more regulatory compositions to activate the T suppressor cells by increasing the acetylation level and/or protein level of FOXP3 in combination with a T suppressor stimulus and/or an antigen. Some methods comprise administering one or more regulatory compositions to activate the T suppressor cells by increasing the acetylation level and/or protein level of FOXP3. Some methods comprise administering soluble GITR or antibodies that bind to GITR ligand. Methods of treating cancer, infectious diseases, and immune deficiency are also disclosed as are vaccination methods. The methods comprise administering one or more regulatory compositions to inactivate the T suppressor cells by reducing the acetylation level and/or protein level of FOXP3. Improved vaccines and vaccination methods are disclosed. Methods of identifying compounds that are useful to modulate acetylation level and/or protein level of FOXP3 and treat diseases are disclosed.

### FIELD OF THE INVENTION

The present invention compositions which can enhance or inhibit suppressor T cell activation and to methods of using such compounds in the treatment of individuals.

### BACKGROUND OF THE INVENTION

This application claims priority to U.S. Provisional Application No. 60/760,549 and U.S. Provisional Application No. 60/794,670, the disclosures of each of which are incorporated herein by reference.

Suppressor T cells are also known as Regulatory T cells in the field. Maintenance of tolerance to self-antigens is essential for the prevention of autoimmunity. Cellular mechanisms for the maintenance of peripheral self-tolerance have been shown to involve deletion of antigen reactive T lymphocytes, clonal anergy, and suppression mediated by suppressor T cells. As indicated by the name, suppressor T cells are able to suppress other immune cells. Activated T cells (CD4+) in the proximity of these suppressor T cells become ‘silenced’ either after cell/cell-contact or by soluble factors released by the suppressor cells (e.g. cytokines). In chronic autoimmune diseases, CD4+ T cells escape the self-tolerance control and mediate immune response to auto-antigens.

Three populations of suppressor T cells have been identified. The Ts1 subset is a natural suppressor cell which expresses CD25 (i.e CD25+) and has biological activity in vitro. The Ts2 subset is a CD25+ suppressor T cell population which can be induced in the periphery by two injections of Mls-1a expressing antigen presenting cells. The third population, Ts3, is CD25− and is induced by multiple injections of irradiated Mls-1a expressing antigen presenting cells. These Ts subsets may be related. The Ts3-like cells have been identified previously and are argued to develop into Ts2 cells in some systems.

In rodents, reduction or functional alteration of CD25+CD4+ regulatory T cells has been shown to cause the spontaneous development of various organ-specific autoimmune diseases including thyroiditis, gastritis, and type 1 diabetes. Regulatory T cells are also critical for the controlled response to environmental antigens and have been shown to prevent inflammatory bowel disease (IBD) as well as allergy.

Each of these three subpopulations of suppressor T cells express FOXP3, a member of the forkhead transcription factor family. Expression of FOXP3 converts naïve T cells towards suppressor T cell and correlates with suppressor activity.

FOXP3, a forkhead protein, is a marker and determinant of regulatory T-cells. Forkhead proteins are a large family of functionally diverse transcription factors that have been implicated in a variety of cellular processes. The name forkhead is derived from the Drosophila melanogaster forkhead(fkh) gene product, which is required for terminal pattern formation in the embryo. FOX (forkhead box) is now used, as the symbol for all chordate forkhead transcription factors. A phylogenetic analysis has resulted in the definition of 15 classes of known FOX proteins; these transcription factors are classified in terms of structure not function. The structure of a forkhead domain bound to DNA has been resolved and resembles a winged helix or butterfly wing. However the forkhead domain of FOXP3 is unusual as it is located near the carboxyl terminus of the protein.

The murine scurfy mutation was found to be a defect in FOXP3. Scurfy is X chromosome linked and murine offspring with the mutation have abnormally active CD4+ cells and autoimmune organ damage resulting in death of hemizygous males early after birth. A similar mutation in humans with a failure to develop CD4+CD25+ cells, called “Immunodysregulation, polyendocrinopathy and enteropathy, X-linked syndrome” (IPEX), also known as “X-linked autoimmunity and allergic dysregulation syndrome” (XLAAD) is a fatal recessive disorder of humans that develops in early childhood. Symptoms are varied and include diarrhea, dermatitis, insulin-dependent diabetes, thyroiditis and anaemia. Massive T cell infiltration into the skin and gastrointestinal tract are also observed. IPEX mutations exist in the forkhead domain of FOXP3, indicating potential disruption of DNA binding. Another study identified a 3-base-pair deletion in the putative leucine-zipper dimerization domain. Therefore mutation in this region could result in aberrant function and a failure to associate with itself or with other members, such as FOXP1 and 4. Little is known about induction or the interactions of the FOXP family members with the transcriptional machinery. TGFb may induce FOXP3 expression and may affect SMAD7 signals that may be relevant. FOX family members may undergo a variety of post-translational modifications permitting or stabilizing interactions with other proteins.

Post translational modifications and interactions of the FOX family members have been studied. The Akt phosphorylation consensus sequence (RXRXXS/THydrophobic) has been defined but is not found in FOXP3. Certain FOX proteins, however, are acted on by AKT. Akt protein kinases, composed of three family members (Akt1, Akt2 and Akt3), regulate a diverse array of cellular functions including apoptosis, proliferation, differentiation, intermediary metabolism and cell size. The FOXO species remains transcriptionally active in the nucleus until acted on by AKT which phosphorylates it. Upon phosphorylation, FOX factors are shuttled from the nucleus to the cytosol in association with 14-3-3. The kinase that phosphorylates FOXP3, if any, has not been identified. In some cases phosphorylation accompanies other modifications such as acetylation.

The Forkhead proteins can associate with discrete histone deacetylases. The deaceytlases can limit acetylation on histones and on certain protein lysine residues. One histone acetyl transferase (HAT) studied is TIP60. Recent studies have indicated that HATs modify a variety of proteins in addition to histone lysines. Transcription factors such as P53 and Myc undergo discrete acetylation by the TIP60 histone acetyl transferase.

Post translational modification may affect function. Although effects on FOXP3 have not been defined to date, Myc is affected by some FOX proteins. FOXO suppresses Myc functions and FOXO dominant negative mutants allow activation of Myc target genes. FOXO mutants which cannot be phosphorylated by AKT abolish a Myc effect of promoting Cyclin D2 transcription and non-phosphorylated FOXO inhibits induction of multiple Myc target genes. These studies indicate that post-translational changes of FOX proteins affect their functions and interactions including modifying Myc targets.

Other types of interactions of forkhead proteins are known. In studies of ectopically over-expressed FOXP 1, 2 and 4 genes, homotypic and heterotypic interactions were noted suggesting that FOXP proteins dimerize at least when overexpressed. In addition, association of FOXP 1 and 2 with a transcriptional co-repressor protein called C-terminal binding protein (CtBP-1) indicates that multimeric complexes of FOX proteins are possible.

There are 5 families of histone acetyl transferases (HATs) and 3 classes of histone deacetylases (HDACs). Both are important enzymes for the transcriptional regulation of gene expression in eukaryotic cells. Chromatin organization involves DNA wound around histone octamers that form nucleosomes which are in turn folded into higher ordered. Core histones contain N-terminal tails that extend from these more compact nucleosomal core particles and it is these tails that are responsible for the interactions that histones participate in during gene regulation.

Deacetylation of epsilon-acetyl-lysine residues within the N-terminal tail of core histones mediates changes in both histone-DNA and histone-non-histone protein interactions. Histone acetylation, which takes place on N-terminal tails of mainly Histones 3 and 4, is generally thought to increase accessibility of transcriptional machinery to promote gene transcription and is mediated by HATs. However, recent work has challenged this hypothesis by showing the opposite effect: histone acetylation actually leads to the repression of many genes, as well as activation of others. Despite conflicting data of the specific function of histone acetylation (repressive vs. permissive), it is a clear regulator of gene transcription and may affect non histone proteins.

Recent studies have indicated that HATs such as TIP60 can also acetylate certain transcriptional proteins, such as Myc and p53 directly and others. Histone acetylases and deacetylases may stably associate with their substrates.

TIP60 associates with Histone deacetylase (HDAC) molecules. TIP60 has been found to associate with class 1 HDAC namely HDAC1 and class 11 histone acetylases such as HDAC7. Interactions of TIP60 with HDAC7 lead to complexes that associate with the class of G-protein coupled endothelin receptors involved in blood vessel reactivity. Under basal conditions the major pools of TIP60 and HDAC7 reside in the nucleus, but surprisingly when endothelin interacts with its receptor, TIP60 associates with HDAC7 and the complex can undergo translocation from the nucleus to the cytoplasm in perinuclear aggregations where it co-localizes with the G-coupled protein receptor.

HDAC molecules themselves, are also able to shuttle between the nucleus and the cytoplasm. Collectively, while it is clear that TIP60 and HDAC 7 molecules are able to translocate to different sites in the cell, the mechanism or modifications that lead to this process are not defined but may include phosphorylation at discrete sites of the HDAC and HAT molecules. HDAC7 is also able to affect central unresponsiveness by inhibiting T cell death. When T cells are activated HDAC7 is exported from the nucleus leading to derepression of Nur77 expression and the induction of negative selection. Therefore, histone acetyltransferases and deacetylases may have multiple functions beyond histone modifications and may affect other proteins. Their expression appears to be directly relevant to the unresponsive phenotype including central and peripheral unresponsiveness. Moreover, this stable and complex arrangement of HAT and HDAC allows for dynamic regulation.

### SUMMARY OF THE INVENTION

The present invention relates to methods for treating individuals who have autoimmune disorders, coronary artery disease, symptoms of allergy, risk of rejection of an allograft, or sepsis/toxic shock. The methods comprise the step of administering to an individual a therapeutically or prophylactically effective amount of one or more regulatory compositions to activate the T suppressor cells by increasing the acetylation level and/or protein level of FOXP3 in combination with a universal T suppressor cell stimulus and/or an antigen that cross reacts with an inflammatory response associated with said autoimmune disorder, coronary artery disease, allergy, allograft rejection, or sepsis/toxic shock, or a nucleic acid molecule that encodes said antigen.

The present invention relates to methods of treating an individual who has an autoimmune disorder, to methods of treating an individual who has an coronary artery disease, to methods of reducing the symptom of allergy of an individual, to methods of reducing the risk of rejection of an allograft in an individual undergoing immunosuppression, and to methods of treating an individual who has or is at an elevated risk of getting sepsis/toxic shock. In some embodiments, the methods comprise the step of administering to the individual a therapeutically or prophylactically effective amount of one or more regulatory compositions to activate the T suppressor cells by increasing the acetylation level and/or protein level of FOXP3. In some embodiments, the methods comprise the steps of removing peripheral blood mononuclear cells (PBMC) from said individual, treating the peripheral blood mononuclear cells with one or more regulatory compositions to activate the T suppressor cells by increasing the acetylation level and/or protein level of FOXP3, and reintroducing treated peripheral blood mononuclear cells to the individual to suppress an aberrant immune response. In some embodiments, the methods comprise the steps of removing peripheral blood mononuclear cells (PBMC) from said individual, isolating T suppressor cells from other PBMC, treating said T suppressor cells with one or more regulatory compositions to activate the T suppressor cells by increasing the acetylation level and/or protein level of FOXP3, and reintroducing treated T suppressor cells to the individual to suppress an aberrant immune response.

The present invention also relates to methods of treating cancer, infectious diseases, and immune deficiency in an individual. The methods comprise the steps of administering to the individual a therapeutically or prophylactically effective amount of one or more regulatory compositions to inactivate the T suppressor cells by reducing the acetylation level and/or protein level of FOXP3.

The present invention relates to methods of treating an individual who has an autoimmune disorder, to methods of reducing the symptom of allergy of an individual, to methods of reducing the risk of rejection of an allograft in an individual undergoing immunosuppression, and to methods of treating an individual who has or is at an elevated risk of getting sepsis/toxic shock. In some embodiments, the methods comprise the step of administering to the individual a therapeutically or prophylactically effective amount of a soluble GITR which binds to GITR ligand.

The present invention relates soluble GITR protein which binds to GITR ligand and to pharmaceutical compositions which comprise a therapeutically or prophylactically effective amount of a soluble GITR which binds to GITR ligand.

The present invention relates antibodies that bind to GITR ligand and to pharmaceutical compositions which comprise a therapeutically or prophylactically effective amount of antibodies that bind to GITR ligand.

The present invention relates to vaccine compositions that comprise a compound that reduces the acetylation level and/or protein level of FOXP3 and methods for vaccinating an individual. The methods comprise administering to an individual a vaccine composition in combination with a compound that reduces the acetylation level and/or protein level of FOXP3.

The present invention relates to compositions useful in the methods of the invention. The present invention also relates to methods of identifying compounds useful for treating an individual who has an autoimmune disorder or treating an individual who has an coronary artery disease or reducing the symptom of allergy of an individual, or reducing the risk of rejection of an allograft in an individual undergoing immunosuppression, or treating an individual who has or is at an elevated risk of getting sepsis/toxic shock. The methods comprise the steps of: performing an assay to determine if a test compound increases acetylation level and/or protein level of FOXP3 in a suppressor T cell; and performing an assay to determine if the test compound that increases acetylation level and/or protein level of FOXP3 in a suppressor T cell is active in an animal model useful to evaluate a compound for activity to treat autoimmune disorder or coronary artery disease or allergy, or reducing the risk of rejection of an allograft, or sepsis/toxic shock.

The present invention also relates to methods of identifying compounds useful for treating cancer, infectious diseases or immune deficiency. The methods comprise the steps of: performing an assay to determine if a test compound decreases acetylation level and/or protein level of FOXP3 in a suppressor T cell; and performing an assay to determine if the test compound that decreases acetylation level and/or protein level of FOXP3 in a suppressor T cell is active in an animal model useful to evaluate a compound for activity to treat cancer, infectious diseases or immune deficiency.

### BRIEF DESCRIPTION OF THE FIGURES

FIGS. 1(A) and (B) contain data from experiments in which HEK 293T cells were transfected with expression plasmids for human FOXP3 and FLAG-tagged TIP60 (FIG. 1(A)) or FOXP3 and FLAG-tagged HDAC7 (FIG. 1(B)) where after RIPA buffer cell lysates were immunoprecipitated (IP) with anti-FLAG M2 mAb, then analyzed by Western blotting with indicated antibodies (IB).

FIGS. 2(A) and (B) contain data from experiments in which U2OS cells were transfected with expression plasmids for GFP-tagged human FOXP3 and, FLAG-tagged TIP60 (FIG. 2(A)), or FLAG-tagged HDAC7 (FIG. 2(B)) where after cells were immunostained with anti-FLAG M2 mAb and detected with Texas Red conjugated anti-mouse mAb. FOXP3 (green) and TIP60 (red) or HDAC7 (red) and subcellular localization were examined under conventional fluorescence microscopy. The blue channel shows the cell nucleus staining with DAPI.

FIG. 3 contains data from Hela cells stably expressing GFP-FOXP3 that were transfected with pCMV-FLAG-tagged TIP60 expression vector where after cells were fixed, then immunostained with anti-FLAG M2 mAb, and detected with Cy5 conjugated anti-mouse mAb (red). Cell nucleus is stained with DAPI as blue color.

FIG. 4 contains data from experiments in which HEK 293T cells were transfected with expression plasmids for human FOXP3 and FLAG-tagged HDAC7 where after RIPA buffer cell lysates were immunoprecipitated (IP) with anti-FLAG M2 mAb, then analyzed by western blotting with indicated antibodies (IB).

FIG. 5(A) shows a schematic representation of FOXP3 series constructs used in cotransfection in HEK293T cells. FIG. 5(B) shows data from experiments in which HEK 293T cells were transfected with expression plasmids for FLAG-tagged HDAC7 and various myc-tagged FOXP3 vectors where after RIPA buffer cell lysates were immunoprecipitated (IP) with anti-FLAG M2 mAb, then analyzed by western blotting with indicated antibodies (IB).

FIG. 6(a) shows a schematic representation of the FORKHEAD domain of FOXP3 binding to the human IL-2 promoter luciferase reporter. FIG. 6(b) shows data from experiments in which Jurkat E6.1 T cells were transfected with the control empty vector (mock), or vectors expressing wild type FOXP3 alone, FOXP3 with different amounts of HDAC7 together, or HDAC7 alone, plus full length IL-2-Luciferase reporter and control TK-Renilla luciferase vector as indicated where after cells were stimulated with 50 ng/ml of PMA and 1 μM ionomycin for 6 hours before lysing and analyzed by means of dual luciferase assay normalized with Renilla luciferase activity.

FIG. 7(a) shows Foxp1, Foxp2, FOXP3 and Foxp4 transcription levels in CD4+CD25+ and CD4+CD25− T cells. FIG. 7(b) shows data from experiment in which HEK 293T cells were cotransfected with expression plasmids for FLAG-tagged FOXP1 (FLAG-FOXP1), myc-tagged wild-type FOXP3 (WT), FOXP3 E251 deletion mutant (delE251) and FOXP3 K250 deletion mutant (delK250) where after cell lysates were immunoprecipitated with anti-FLAG M2 mAb, then analyzed by Western blotting with anti-myc-tag 4E10 mAb, or anti-FLAG M2 mAb. FOXP3 and actin expression levels in cell lysates were analyzed with anti-myc or anti-actin antibodies.

FIG. 8(a) shows nuclear extracts from primary normal and XLAAD patient (delE251) T cell lines that were immunoblotted with anti-human FOXP3 monoclonal antibody 221D. FIG. 8(b) shows a schematic representation of the primers used for detection on human IL-2 promoter region. FIG. 8(c) shows chromatin immunoprecipitation results.

FIG. 9 shows results of immunoblots demonstrating that FOXP3 is acetylated in human CD4+CD25+T cells. Nuclear extracts from Jurkat E6.1 T cells and human CD4+CD25+T cells were immunoprecipitated with anti-FOXP3 hFOXY, or control IgG, then analyzed by western blotting with rabbit anti-acetyl-lysine (Upstate) (top panel) and reprobed with anti-FOXP3 221D (bottom panel).

FIG. 10 shows results from experiments described in example 2. Splenocytes from collagen induced arthritic mice treated with VPA (no disease evident) or PBS (disease evident) were stained for cell-surface CD25 and intracellular FOXP3. The CD25+FOXP3+cell subpopulation was gated, and FOXP3 level was analyzed.

FIG. 11, panels A-F show that FOXP3 is acetylated and that the acetylation is promoted by TIP60. FIG. 11 panel A shows TIP60 expression in both human CD4+CD25+ T cells and CD4+CD25− T cells. FOXP3 and β-actin expression levels were also analyzed by immunoblotting with 221D and anti-β-actin antibodies. FIG. 11 panel B shows nuclear co-localization of endogenous FOXP3 with TIP60 in CD4+CD25+ T cells. Human CD4+CD25+ T cells were stimulated for 2 hrs with PMA/ionomycin, fixed, permeabilized and stained for human FOXP3 by mouse anti-human FOXP3 hFOXY (eBioscience), in conjunction with rabbit anti-TIP60 (Upstate) as indicated. Cell nucleus was demonstrated by DAPI staining. FIG. 11 panel C shows FOXP3 associates with TIP60 in vivo. HEK 293T cells were cotransfected with expression plasmids for FLAG-TIP60, or HA-FOXP3a as indicated, immunoprecipitated with anti-FLAG M2, then analyzed by Western blotting with anti-FOXP3 221D, or anti-FLAG M2. FIGS. 11D and 11E show that TIP60 promotes FOXP3 acetylation. In FIG. 11 panel D, HEK 293T cells were cotransfected with HA-FOXP3a and an increasing amount of FLAG-TIP60 as indicated, then immunoprecipitated either with acetylated-lysine Ac-K-103 (top panel) or with anti-HA F-7 probe (bottom), following by Western blotting with HRP-conjugated anti-HA 3F10. In FIG. 11 panel E, HEK 293T cells were cotransfected with HA-FOXP3a and FLAG-TIP60 as indicated, then immunoprecipitated with anti-HA F-7 probe, following by Western blotting either acetylated-lysine (Cell Signaling #9441, left panel) or HRP-conjugated anti-HA 3F10 (right panel). FIG. 11 panel F shows that FOXP3 is acetylated in human CD4+CD25+ T cells. Nuclear extracts from Jurkat E6.1 T cells and human CD4+CD25+ T cells were immunoprecipitated with anti-FOXP3 hFOXY, or control IgG, then analyzed by western blotting with rabbit anti-acetyl-lysine (Upstate) (top panel) and reprobed with anti-FOXP3 221 D (bottom panel).

FIG. 12 panels A-C show that FOXP3 associates with HDAC7 in primary CD4+CD25+ T cells. FIG. 12 panel A, shows a schematic representation of the myc-tagged FOXP3 constructs used for detection of FOXP3-HDAC7 association. FIG. 12 panel B shows that FOXP3 associates with HDAC7. HEK 293T cells were cotransfected with expression plasmids for FLAG-tagged HDAC7 (FLAG-HDAC7), myc-tagged FOXP3a (3a), or myc-tagged FOXP3b lacking exon 2 (3b) as indicated. Cell lysates were either immunoprecipitated with anti-FLAG M2 then analyzed by Western blotting with HRP-conjugated 4E10 (top panel) and reprobed with anti-FLAG M2 (bottom panel), or immunoprecipitated with anti-myc-tag 4E10, then immunoblotted with anti-FLAG M2 (third panel) and reprobed with HRP-conjugated 4E10 (second panel). FIG. 11 panel C shows that endogenous FOXP3 associates with HDAC7 in human CD4+CD25+ T cells. Nuclear extracts from Jurkat E6.1 T cells and human CD4+CD25+ T cells were immunoprecipitated with anti-HDAC7 C-18, or control IgG, then analyzed by western blotting with anti-FOXP3 221D (top panel) and reprobed with anti-HDAC7 KG-17 (bottom panel).

FIG. 13 panels A-D show N-terminal 106-190aa as the transcriptional repression domain of FOXP3 is essential for TIP60 and HDAC7 association. FIG. 13 panel A shows a schematic representation of FOXP3 binding to 8× Forkhead binding sites luciferase reporter construct (8× FK1TK-Luc) used in luciferase reporter assay. FIG. 13 panel B shows data from a luciferase reporter assay using 8× FK1TK-Luc reporter. 293T cells were transfected with the control empty vector (mock), wild type FOXP3a, FOXP3b, FOXP3 forkhead domain deletion (N1) expression vectors, or FOXP3 deletion mutant del C4 or delC3, plus 8× FK1TK-Luc luciferase reporter and control TK-Renilla luciferase vector as indicated, then analyzed by means of dual luciferase assay normalized with Renilla luciferase activity. Results are means of 3 separated experiments with SD. FIG. 13 panel C shows that HDAC7 associates with 3a, N1, and C4, but not C3 with an additional deletion of 106-190aa region. 293T cells were transfected with a panel of myc-tagged FOXP3 expression vectors, including 3a (wild type), deletion Mutants N1, C1, C3, C4, combined with FLAG-HDAC7 as indicated, immunoprecipitated with anti-FLAG M2, then analyzed by Western blotting with anti-myc 9E10 (top panel), or anti-FLAG M2(second panel). Cell lysates were analyzed by immunoblotting with anti-myc 9E10 (third panel), and control anti-B-actin (bottom panel). FIG. 13 panel D shows that TIP60 associates with 3a, 3b, delE, delK, N1, but not C3 with an additional deletion of 106-190aa region. 293T cells were transfected with a panel of myc-tagged FOXP3 expression vectors, including 3a, 3b, deletion mutant delE (E251 deleted), delK (K250 deleted), N1, C1, C3, with or without FLAG-TIP60 as indicated, immunoprecipitated with anti-FLAG M2, then analyzed by Western blotting with HRP conjugated anti-FLAG M2 (top Panel) or HRP-9E10 (middle panel); Cell lysates were also immunoprecipitated with anti-myc 9E10, then analyzed by HRP-9E10 (bottom panel).

FIG. 14 panels A-D shows that FOXP3 mediates transcriptional repression via the forkhead domain as part of an ensemble with HDAC7 and TIP60. FIG. 14 panel A shows a schematic representation of GAL4-FOXP3 binding to 5× GAL4 binding sites luciferase reporter construct (pG5Luc) used in luciferase reporter assay. FIG. 14 panel B, shows data from a luciferase reporter assay using pG5Luc reporter with overexpression of TIP60. 293T cells were transfected with the control pBIND empty vector (pBIND), pBIND-FOXP3a, pBIND-FOXP3a and pFLAG-TIP60 or, pBIND-FOXP3qa and the HAT-deficient TIP60 expressing construct (pFLAG-MUT-TIP60), plus pG5Luc luciferase reporter and control MSV-β-Gal as indicated, then analyzed by means of luciferase assay normalized with β-Gal activity. Results are means of 3 separated experiments with SD. FIG. 14 panel C, shows data from a Luciferase reporter assay using pG5Lue reporter with knockdown of endogenous TIP60. 293T cells were transfected with the control pBIND empty vector (pBIND) and non-target control shRNA (lane 1), pBIND-FOXP3a and non-target control shRNA (lane 2), or pBIND-FOXP3a and TIP60 shRNA sh15, plus pG5Luc luciferase reporter and control MSV-β-Gal as indicated. 72 hours after the transfection, cell lysates were analyzed by means of luciferase assay normalized with β-Gal activity. Results are means of 3 separated experiments with SD. The knockdown efficiency of TIP60 shRNA was evaluated by western blotting with Rabbit anti-TIP60, and the same membrane was reprobed with anti-α-tubulin monoclonal Ab. FIG. 14 panel D, shows data from Two million transfected Jurkat E6.1 T cells by electroporation with control empty vectors or FOXP3a (10 μg) and FOXP3b (10 μg), and various combinations of TIP60 and HDAC7 expressing plasmids as indicated were stimulated respectively with plate-bound TCR Vβ 8.1 plus soluble anti-CD28 in two ml cultured medium for 24 hours. IL-2 production in cultured medium was diluted 4 times with ELISA blocking buffer, then measured with a commercial obtained IL-2 ELISA kit (eBioscience). The repression efficiency of the empty vector transfected sample was defined as zero, and the one with 10 μg of each FOXP3a, FOXP3b, TIP60 and HDAC7 plasmids transfected sample with the maximal repression efficiency was defined as one hundred percent. The result is the average standard error by mean of three independent experiments. The inserted figure is one representative result of three independent experiments showing the actual amount of IL-2 production after TCR plus CD28 stimulation in Jurkat T cells, which were co-transfected with either 10 μg of each FOXP3a, TIP60 and HDAC7 expressing plasmids, or equal amounts of empty vectors (mock). Transfection of the HAT-deficient TIP60 or HDAC deficient HDAC7 species led to less inhibition of 1L2 production.

FIG. 15 panels A and B show that T cell stimulation antagonizes FOXP3 recruiting HDAC9. In FIG. 15 panel A, HA-FOXP3a transfected Jurkat E6.1 T cells (10×106) were not stimulated, or stimulated with plate-bound TCR Vβ 8.1 plus soluble anti-CD28 for 4 hours (with or without 400 nM Trichostatin A, indicated above lanes), lysed, and equal nuclear extracts were immunoprecipitated with anti-HA probe (F-7), then analyzed for FOXP3-HDAC9 association by immunoblotting with rabbit anti-HDAC9 antibody H-45 (lane 3-6). The input nuclear extracts of TSA treated cells, with or without stimulation, were also immunoblotted with anti-HDAC9 H-45 (lane 1-2). In FIG. 15 panel B, in vitro activated and expanded human CD4+CD25+ T cells were treated with or without 400 nM Trichostatin A, lysed and equal nuclear extracts were immunoprecipitated with anti-FOXP3 mAb 221D or control IgG, then analyzed for endogenous FOXP3-HDAC9 association by immunoblotting with rabbit anti-HDAC9 H-45 (lane 3,4,5; right panel). The input nuclear extracts of TSA treated or non-treated cells were also immunoblotted with anti-HDAC9 Ab H-45 to reveal total HDAC9 expression level (lane 1 and lane 2, left panel).

### DETAILED DESCRIPTION OF PREFERRED EMBODIMENTS

Increasing the acetylation level and/or protein level of FOXP3 activates T suppressor cells while reducing the acetylation level and/or protein level of FOXP3 inactivate the T suppressor cells. Accordingly, treatment of diseases and conditions associated with undesirably high levels of activated CD4+ T cells, such as autoimmune diseases, coronary artery disease, allergies and allograft rejection, allergies, sepsis/toxic shock by activation of T suppressor cells can be achieved by increasing the acetylation level and/or protein level of FOXP3. Treatment of diseases and conditions associated with undesirably low levels of activated CD4+ T cells, such as in patients with cancer or at an elevated risk of developing cancer, by deactivation of T suppressor cells can be achieved by reducing the acetylation level and/or protein level of FOXP3.

While the present invention is not intended to be limited to any theory or proposed mechanism, FOXP3 might associate with acetyltransferases/deacetylases and undergo acetylation changes itself as one form of post-translational modification. It is conceivable that phosphorylation of certain serine or threonine residues on the forkhead domain protein may be permissive for subsequent acetylation/deacetylation events mediated by complexed acetyl transferases and deacetylases. It is also possible that deacetylases associate with FOX proteins and that discrete, sequential or complex acetylation patterns may be a general mechanism to influence transcriptional factors.

Increasing the Acetylation Level and/or Protein Level of FOXP3 Activates T Suppressor Cells

In order to decrease levels of activated CD4+ T cells in diseases characterized by undesirably high levels of activated CD4+ T cells, T suppressor cells are activated by increasing the acetylation level and/or protein level of FOXP3.

According to some embodiments, methods are provided for treating an individual who has an autoimmune disorder. According to some embodiments, methods are provided for treating an individual who has coronary artery disease. According to some embodiments, methods are provided for reducing the symptom of allergy of a subject. According to some embodiments, methods are provided for reducing the risk of rejection of an allograft in a subject undergoing immunosuppression. According to some embodiments, methods are provided for treating or reducing the risk of sepsis/toxic shock.

Methods comprise the step of administering to a subject a therapeutically or prophylactically effective amount of regulatory composition to activate the T suppressor cells by increasing the acetylation level and/or protein level of FOXP3. In some embodiments, the composition is delivered directly to the individual systemically or at a specific site targeted for delivery. In some embodiments, peripheral blood mononuclear cells (PBMC) are removed from a patient, the peripheral blood mononuclear cells are treated with regulatory composition; and the treated peripheral blood mononuclear cells are reintroduced to the patient. Aberrant immune responses are thereby suppressed. In some embodiments, peripheral blood mononuclear cells (PBMC) are removed from a patient, T suppressor cells are isolated from other PBMC; the T suppressor cells are treated with regulatory composition; and the treated T suppressor cells are reintroduced to the patient. Aberrant immune responses are thereby suppressed.

According to some embodiments, methods include administering one or more agents that function as universal T suppressor cell stimulus. Such agents function to activate the T suppressor cells. An example of universal T suppressor cell stimulus is anti-CD3 antibodies. The anti-CD3 antibodies and/or nucleic acid molecules that encode them are administered to the individual in combination with one or more regulatory composition that activate the T suppressor cells by increasing the acetylation level and/or protein level of FOXP3. Such combinations may be delivered simultaneously or at different times. In some embodiments, the universal T suppressor cell stimulus is anti-CD3 antibodies in combination with IL-2 and/or nucleic acid molecules that encode them. In some embodiments, In some embodiments, In some embodiments, the universal T suppressor cell stimulus is anti-CD28 antibodies and/or nucleic acid molecules that encode them. In some embodiments, the combinations may be delivered sequentially.

According to some embodiments, methods include administering one or more antigens that cross reacts with an inflammatory response (i.e. cells and/or antibodies that are involved in an immune response) associated with said autoimmune disorder, coronary artery disease, allergy, allograft rejection, or sepsis/toxic shock, and/or one or more nucleic acid molecules that encode one or more such antigens. The antigen(s) and/or nucleic acid molecule(s) are administered to the individual in combination with one or more regulatory composition that activate the T suppressor cells by increasing the acetylation level and/or protein level of FOXP3. Such combinations may be delivered simultaneously or at different times. In some embodiments, the combinations may be delivered sequentially.

Some embodiments include to pharmaceutical compositions which comprise therapeutically effective amounts of one or more antigen(s) and/or nucleic acid molecule(s) in combination with one or more regulatory composition that activate the T suppressor cells by increasing the acetylation level and/or protein level of FOXP3 and optionally, one or more immunosuppressants that do not activate the T suppressor cells by increasing the acetylation level and/or protein level of FOXP3. Some embodiments include to pharmaceutical kits which comprise a package that contains a first container that comprises one or more one or more antigen(s) and/or nucleic acid molecule(s) and a second container that comprises one or more regulatory composition that activate the T suppressor cells by increasing the acetylation level and/or protein level of FOXP3, and optionally a third contain that contains one or more immunosuppressants that do not activate the T suppressor cells by increasing the acetylation level and/or protein level of FOXP3. The containers may be for example separate containers provided as or within a single package, a single container or package with separate compartments such as a blister pack that has separate compartments.

The antigens are associated with autoimmunity generally include cell receptors and cells which produce “self”-directed antibodies as well as antibodies themselves. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases. Antigens may comprise epitopes that are identical to or crossreactive with epitopes of the variable region of the T cell receptors. In RA, several specific variable regions of T cell receptors (TCRs) which are involved in the disease have been characterized. These TCRs include Vβ-3, Vβ-14, Vβ-17 and Vα-17. In MS, several specific variable regions of TCRs which are involved in the disease have been characterized. These TCRs include Vβ-7 and Vα-10. In scleroderma, several specific variable regions of TCRs which are involved in the disease have been characterized. These TCRs include Vβ-6, Vβ-8, Vβ-14 and Vα-16, Vα-3C, Vα-7, Vα-14, Vα-15, Vα-16, Vα-28 and Vα-12. In order to treat patients suffering from a T cell mediated autoimmune disease, particularly those for which the variable region of the TCR has yet to be characterized, a synovial biopsy can be performed. Samples of the T cells present can be taken and the variable region of those TCRs identified using standard techniques. In some embodiments, the antigen is a T cell receptor or a fragment thereof that comprises a variable region including at least one epitope. In some embodiments, the antigen is a T cell receptor on a killed or inactivated cell such as ultraviolet treated T cells such as for example those taken from the patient. B cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. Each of these diseases is characterized by antibodies which bind to endogenous antigens and initiate the inflammatory cascade associated with autoimmune diseases. Antigens may comprise an epitope that is identical to or cross reactive with an epitope of a variable region of an antibodies. In some embodiments, the antigen is an antibody or a fragment thereof that comprises a variable region including at least one epitope. In order to treat patients suffering from a B cell mediated autoimmune disease, the variable region of the antibodies involved in the autoimmune activity must be identified. A biopsy can be performed and samples of the antibodies present at asite of inflammation can be taken. The variable region of those antibodies can be identified using standard techniques. In the case of SLE, one antigen is believed to be DNA. Thus, in patients to be treated for SLE, their sera can be screened for anti-DNA antibodies and an antigen can be prepared which includes the variable region of such anti-DNA antibodies found in the sera.

The collagen used in experiments with CIA model was collagen from chicken sternal cartilage powder, Type II, from Sigma Cat#C9301. The peptide that was used in EAE experiments was Mouse MOG38-50 peptide (GWYRSPFSRVVHL-SEQ ID NO:1), which was synthesized using>F-moc solid phase methods and purified through HPLC by Invitrogen Life Technologies.

In some embodiments, antigens may be allergens, proteins associated with coronary artery disease, proteins of grafted tissue or cells, or the variable regions of receptors and antibodies involved in immune responses associated with sepsis/toxic shock.

In some embodiments, one or more universal T suppressor cell stimulus, for example in protein and/or DNA form, is administered in combination with one or more antigens, for example in protein and/or DNA form, that cross reacts with an inflammatory response (i.e. cells and/or antibodies that are involved in an immune response) associated with said autoimmune disorder, coronary artery disease, allergy, allograft rejection, or sepsis/toxic shock, and/or one or more nucleic acid molecules that encode one or more such antigens.

According to some embodiments, methods include administering one or more immunosuppressants that do not activate the T suppressor cells by increasing the acetylation level and/or protein level of FOXP3, in combination with one or more regulatory composition that activate the T suppressor cells by increasing the acetylation level and/or protein level of

FOXP3. Examples of immunosuppressants include but are not limited to corticosteroids, rapamycin, Azathioprine (Imuran), Mycophenolate (MFM or CellCept), Cyclosporine (Sandimmune), Mercaptopurine (6-MP), basiliximab, daclizumab, sirolimus, tacrolimus, Muromonab-CD3, cyclophosphamide, and methotrexate. Such combinations may be delivered simultaneously or at different times. In some embodiments, the combinations may be delivered sequentially. Some embodiments include to pharmaceutical compositions which comprise therapeutically effective amounts of one or more immunosuppressants that do not activate the T suppressor cells by increasing the acetylation level and/or protein level of FOXP3 in combination with one or more regulatory composition that activate the T suppressor cells by increasing the acetylation level and/or protein level of FOXP3. Some embodiments include to pharmaceutical kits which comprise a package that contains a first container that comprises one or more immunosuppressants that do not activate the T suppressor cells by increasing the acetylation level and/or protein level of FOXP3 and a second container that comprises one or more regulatory composition that activate the T suppressor cells by increasing the acetylation level and/or protein level of FOXP3. The containers may be for example separate containers provided as or within a single package, a single container or package with separate compartments such as a blister pack that has separate compartments.

Acetylation levels and/or protein levels are increased by administration of regulatory compositions that comprises one or more deacetylase inhibitors, and/or one or more acetyl transferases enhancers and/or one or more reagents that change the expression level or activity of TIP60 and/or the expression level or activity of HDAC7.

According to some embodiments, the regulatory composition comprises one or more deacetylase inhibitors. Examples of deacetylase inhibitor include, but are not limited to the group consisting of trichostatin A, trapoxin B, butyrates (e.g., sodium butyrate, sodium phenylbutyrate, arginine butyrate, and butyric acid), MS 275-27 (Mitsui Pharmaceuticals, which is disclosed in U.S. Published Application Number 20040186049 and 20050176686, which is incorporated herein by reference) m-carboxycinnamic acid bis-hydroxamide, depudecin, oxamflatin, apicidin, suberoylanilide hydroxamic acid, Scriptaid (which is also disclosed in U.S. Published Application Number 20040186049 and 20040077591), pyroxamide, valproic acid, 2-amino-8-oxo-9,10-epoxy-decanoyl, 3-(4-aroyl-1H-pyrrol-2-yl)-N-hydroxy-2-propenamide, CI-994 (Pfizer), Pivanex (Titan Pharmaceuticals; which is disclosed in U.S. Published Application Number 20050176686, which is incorporated herein by reference), FK228(which is also disclosed in U.S. Published Application Number 20040077591), NVP-LAQ824 (Novartis, which is also disclosed in U.S. Published Application Number 20050176686), NVP-LBH589 (Novartis), PXD101 (Prolifix, Curagen, TopoTarget), and FR901228 (depsipeptide-Fujisawa, which is also disclosed in U.S. Published Application Number 20040186049 and 20040077591). Combinations of regulatory compositions may be delivered simultaneously or at different times. In some embodiments, the combinations may be delivered sequentially.

According to some embodiments, the regulatory composition comprises one or more acetyl transferases enhancers.

According to some embodiments, the regulatory composition comprises one or more reagents that change the expression level or activity of TIP60 and/or the expression level or activity of HDAC7. Examples of reagents that change the expression level or activity of TIP60 include antisense, RNAi and siRNA reagents which inhibit TIP60 expression. Examples of reagents that change the expression level or activity of HDAC7 include antisense, RNAi and siRNA reagents which inhibit HDAC7 expression.

According to some embodiments, the regulatory composition comprises one or more deacetylase inhibitors in combination with one or more acetyl transferases enhancers in combination with one or more reagents that change the expression level or activity of TIP60 and/or the expression level or activity of HDAC7.

Diseases and conditions in which activated CD4+ T cells are undesirable and therefore in which methods of decreasing levels of activated CD4+ T cells include but not limited to coronary artery disease, autoimmune diseases, cell, tissue and organ transplantation procedures which lead to rejection and/or graft versus host disease, allergies and allergic reactions, and sepsis/toxic shock. Examples of autoimmune diseases include but are not limited to autoimmune diseases include Rheumatoid arthritis (RA), osteoarthritis, multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease, Hashimoto's disease, inflammatory bowel disease, scleroderma, oophoritis, ulcerative colitis, Lupus (SLE), Grave's disease, myasthenia gravis, autoimmune hemolytic anemia, autoimmune thrombocytopenia, asthma, cryoglobulinemia, primary biliary sclerosis, dermatomyositis, pemphigus vulgaris, myasthenia gravis, hemolytic anemia and pernicious anemia. Examples of cell, tissue and organ transplantation procedures include but are not limited to therapeutic cell transplants such as stem cells, muscle cells such as cardiac cells, islet cells, liver cells, bone marrow transplants, skin grafts, bone grafts, lung transplants, kidney transplants, liver transplants, and heart transplants.

Reducing the Acetylation Level and/or Protein Level of FOXP3 Inactivate the T Suppressor Cells.

In order to increase levels of activated CD4+ T cells in diseases characterized by undesirably low levels of activated CD4+ T cells, T suppressor cells are deactivated by reducing the acetylation level and/or protein level of FOXP3.

Diseases and conditions in which inactivate CD4+ T cells are undesirable and therefore in which methods of increasing levels of activated CD4+ T cells include infectious diseases, cancer, and immune deficiency/immunocompromised patients. Examples of infectious diseases relate to various pathogen infections such as viral, bacterial, mycoplasm, and infections by unicellular and multicellular eukaryotic organisms. Common human pathogens include but are not limited to HIV, HSV, HPV, Hepatitis A, B and C viruses, influenza, denge, zostrella, rubella, RSV, rotavirus, gram positive, gram negative, streptococcus, tetanus, staphalococcus, tuberculosis, listeria, and malaria. Examples of cancer include but are not limited to lymphomas, leukemia, melanomas, adenocarcinomas, blastomas and sarcomas. Human cancers include but are not limited to skin, brain, bone, liver, lung, colon, kidney, bladder, pancreatic, muscle, and cartilage. Patients who are immune deficient or immunocompromised may be in such condition due to a disease or infection or as a side effect of treatment for another condition. HIV infected patients and burn victims are often immune deficient. Patients who have had bone marrow transplant procedures are often immunocompromised.

### Vaccine Enhancer

In addition, reducing the acetylation level and/or protein level of FOXP3 to deactivate T suppressor cells is useful in vaccine protocols. Thus, inhibition of HAT activity such as TIP60 activity or inhibition of HAT expression such as TIP60 expression such as by use of antisense of siRNA technology reduces FOXP3 acetylation level and/or protein levels and thus leads to deactivate T suppressor cells. Enhanced immune response are induced by vaccines administered in combination with compositions that inhibit HAT activity or expression.

Examples of vaccines include DNA vaccines, recombinant vector vaccines, killed or inactivated pathogen-based vaccines, subunit vaccines and cancer cell vaccines.

DNA vaccines are described in U.S. Pat. Nos. 5,593,972, 5,739,118, 5,817,637, 5,830,876, 5,962,428, 5,981,505, 5,580,859, 5,703,055, 5,676,594, which are each incorporated herein by reference. Examples of attenuated live vaccines and those using recombinant vectors to deliver foreign antigens are described in U.S. Pat. Nos. 4,722,848; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; and 5,482,713, which are each incorporated herein by reference. examples of cancer vaccines are disclosed in Berd et al. May 1986 Cancer Research 46:2572-2577 and Berd et al. May 1991 Cancer Research 51:2731-2734, which are incorporated herein by reference.

Vaccines against infectious agents and methods of treating and preventing infections are well known. Antigens useful in such compositions and methods include antigens that have epitopes identical to or cross reactive with epitopes from pathogen antigens. In some embodiments, the antigen is a pathogen antigen or fragment thereof. In some embodiments, the antigen is an attenuated or killed pathogen.

Some embodiments of the invention relate to cancer vaccines and methods of treating cancer. Antigens useful in such compositions and methods include antigens that have epitopes identical to or cross reactive with epitopes from cancer cells. In some embodiments, the antigen is a cancer specific protein or fragment thereof. In some embodiments, the antigen is a tissue specific protein or fragment thereof of the tissue from which the cancer is derived. In some embodiments, the antigen is an inactivated or killed cancer cell such as cancer cells treated with ultraviolet radiation.

Examples of oncogenes include myb, myc, fyn, bcr/abl, ras, src, P53, neu, trk and EGRF. In addition to oncogene products as target antigens, variable regions of antibodies made by B cell lymphomas and variable regions of T cell receptors of T cell lymphomas can also be used as target antigens. Additionally, other tumor-associated proteins can be used as target proteins. Such proteins are generally those which are found at higher levels in tumor cells. Examples include CEA and the protein recognized by monoclonal antibody 17-1A and folate binding proteins. While the present invention may be used to immunize an individual against one or more of several forms of cancer, the present invention is particularly useful to treat an individual who has cancer or to immunize an individual who is predisposed to develop a particular cancer or who has had cancer and is therefore susceptible to a relapse.

### Formulations, Doses and Treatment Regimens

Pharmaceutical compositions may be formulated by one having ordinary skill in the art with compositions selected depending upon the chosen mode of administration. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985) a standard reference text in this field, which is incorporated herein by reference. Pharmaceutically acceptable carriers are those that are compatible with the other ingredients in the formulation and biologically acceptable.

The pharmaceutical compositions of the present invention may be administered by any means that enables the active agent to reach the agent's site of action in the body of a mammal. Pharmaceutical compositions may be administered orally or parenterally, i.e., intratumor, intravenous, subcutaneous, intramuscular, etc. The compounds of this invention may be administered neat or in combination with conventional pharmaceutical carriers, the proportion of which is determined by the solubility and chemical nature of the compound, chosen route of administration and standard pharmacological practice. The pharmaceutical carrier may be solid or liquid.

Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions which are sterile solutions or suspensions can be administered by, for example, intramuscular, intraperitoneal or subcutaneous injection. Sterile solutions can also be administered intravenously. Oral administration may be either liquid or solid composition form.

The compounds of this invention may be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. The dosage requirements vary with the particular compositions employed, the route of administration, the severity of the symptoms presented and the particular subject being treated. Based on the results obtained in the standard pharmacological test procedures, projected daily dosages of active compound would be 0.02 μg/kg-750 μg/kg. Treatment will generally be initiated with small dosages less than the optimum dose of the compound. Thereafter the dosage is increased until the optimum effect under the circumstances is reached; precise dosages for oral, parenteral, nasal, or intrabronchial administration will be determined by the administering physician based on experience with the individual subject treated.

### Blocking GITR Ligand Binding by Activated T Cells Inhibits Resistance to Suppression

Glucocorticoid-Induced Tumor Necrosis Factor Receptor (GITR) is expressed on CD4⁺CD25⁺ and activated CD4⁺CD25− T cells. Antigen-presenting cells (APCs) constitutively express GITR ligand (GITR-L) which can be down-regulated by inflammatory stimuli through TLR signaling pathways. The engagement of GITR on CD4⁺CD25⁻ T effectors cells but not CD4⁺CD25⁺ Tregs by GITR-L on APCs renders the activated T effector cells refractory to suppression. If soluble GITR (sGITR) that can bind to GITR ligand+ cells is present, the sGITR competes with CD4⁺CD25⁻ GITR+ cells and thereby prevents the CD4⁺CD25⁻ GITR+ cells from binding to GITR ligand+ cells which results in the CD4⁺CD25⁻ GITR+ activated T effector cells not becoming resistant to suppression. Suppression of CD4⁺CD25⁻ GITR+ activated T effector cells is desirable in the treatment of autoimmune diseases and transplantations because the immune system is system down-modulated. Thus sGITR is useful or prevent autoimmune diseases, allergies, inflammation, septic/toxic shock and in transplantation procedures.

Mouse GITR was originally cloned using differential display to identify T-cell mRNAs induced by the synthetic glucocorticoid hormone dexamethasone. Human GITR was cloned soon after, based on sequence-specific homology and motif search. Stimulation of CD4⁺CD25⁺ Tregs with anti-GITR breaks immunological self-tolerance. Mouse GITR ligand (GITR-L) is a costimulator for T cells, and its interaction with G1TR reverses suppression by CD4⁺CD25⁺ T cells. Ligation of GITR on CD4⁺CD25^{S} effector T cells but not CD4⁺CD25⁺ Tregs was required to abrogate suppression and suggested that GITR/GITR-L engagement provides a previously undefined signal that renders effector T cells resistant to the inhibitory effects of CD4⁺CD25⁺ T cells. The extracellular region of GITR has one conserved cysteine-rich repeat, which is a common feature shared with other TNFR family members. However, there are at least four different GITR isoforms expressed in mouse T cells. One isoform in the mouse, named GITR-D, is a secreted protein lacking a transmembrane and intracellular domain, which can bind the GITR-L and function as a decoy receptor. Interestingly, this soluble GITR (sGITR) stimulates osteoclast differentiation at least in in vitro culture systems.

Soluble murine GITR are reported in Nocentini, G. et al. (2000) Cell Death and Differentiation 7:408-410, which is incorporated herein by reference. A human GITR-D is disclosed in Genbank accession number AAF63506 and coding sequence in Genbank accession number AF241229. Human secreted GITR mRNA is disclosed in Genbank accession number NM 148901, which is incorporated herein by reference. Human GITR protein sequences are disclosed in Genbank accession numbers AAD22635, Q9Y9U5, NP004186, NP683699, and NP683700, which are each incorporated herein by reference. Human GITR nucleic acid sequences are disclosed in Genbank accession numbers AF125304, NM004195, NM148901, and NM 148902, which are each incorporated herein by reference. Soluble GITR proteins are those fragments of GITR which retain their ability to bind to GITR ligand+ cells, preferably those which are free of all or part of the transmembrane domain of the full length protein.

A human sGITR isoform was cloned from in vitro expanded human CD4⁺CD25⁻ T cells. This demonstration of a sGITR isoform supports a role in the negative feedback pathway in Treg cell biology. While not being limited to any particular theory, it appears that signals or surface interactions of activated CD4⁺CD25⁺ Tregs, as a result of their own association with certain APCs, may lead to their own expression of sGITR or induce CD4⁺CD25⁻ effector T cells to express sGITR. The secretion of this soluble form into the microenvironment allows for its association with GITR-L on APCs. Normally GITR-L on the APC would render activated effector T cells refractory to suppression. However, when blocked by sGITR, this refractory state would not occur, and the cells would be suppressible. This soluble form of GITR would act much like the analogous osteoprotegerin in the receptor activator of NFKB (RANK) system to diminish the costimulation function of GITR-L on T cells.

The ability of suppressor cells to affect some functions and not others may relate to molecules such as secreted GITR forms or other yet to be described phenotype-modifying molecules. Clearly, other secreted molecules made by Tregs, or even molecules that are induced by Foxp3 expression to become cell surface expressed, may play a role in mediating suppression by disabling the APCs ability to induce a ‘suppressive phenotype on effector cells in the lymph node.

Soluble GITR proteins may be prepared using virtually any of several well-known techniques for the preparation of proteins. For example, the proteins may be prepared using conventional solution or solid phase peptide syntheses. Suitable procedures for synthesizing proteins are well known in the art.

The protein may also be synthesized using conventional recombinant genetic engineering techniques. For recombinant production, a polynucleotide sequence encoding the protein is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the protein. Depending on the expression system used, the expressed protein is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems may be utilized to express the protein described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The protein can be purified by art-known techniques such as high performance liquid chromatography, ion exchange chromatography, gel electrophoresis, affinity chromatography and the like. The actual conditions used to purify the protein will be apparent to those having skill in the art.

For affinity chromatography purification, any antibody which specifically binds the protein may be used. For the production of antibodies, various host animals, including but not limited to rabbits, mice, rats, etc., may be immunized by injection with protein. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to the protein may be prepared using any technique which provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce specific single chain antibodies.

In addition to making antibodies against GITR, antibodies, including monoclonal, chimerized, or humanized antibodies against GITR-L can be made and used as therapeutic compositions to treat autoimmuine diseases, allergies, inflammation, septic/toxic shock and in transplantation procedures. Individuals are administered antibodies in therapeutically effective amounts to down-modulate the immune system.

Aspects of the present invention thus include preventing or inhibiting the process by which effector cells become refractory to suppression by binding to GITR-L with either GITR or a GITR-L-binding fragment thereof or an antibody that binds to GITR-L or a GITR-L-binding fragment thereof. The GITR protein or a GITR-L-binding fragment thereof, or the antibody that binds to GITR-L or a GITR-L-binding fragment thereof competes with and thereby reduces the binding of GITR+ cells to GITR-L+ cells.

The methods of the invention may. include the use one or more compounds selected from the group consisting of: soluble GITR proteins that bind to GITR ligand and antibodies that bind to GITR ligand. Such agents, if used in combination, may be formulated separately or in combination. Other compounds which can be further used in the methods of the invention include one or more deacetylase inhibitors and/or compounds that inhibit expression of a deacetylase. Deacetylase inhibitors may be selected from the group consisting of trichostatin A, trapoxin B, butyrates (e.g., sodium butyrate, sodium phenylbutyrate, arginine butyrate, and butyric acid), MS 275-27, m-carboxycinnamic acid bis-hydroxamide, depudecin, oxamflatin, apicidin, suberoylanilide hydroxamic acid, Scriptaid, pyroxamide, valproic acid, 2-amino-8-oxo-9,10-epoxy-decanoyl, 3-(4-aroyl-1H-pyrrol-2-y1)-N-hydroxy-2-propenamide, C1994, Pivanex, FK228, NVP-LAQ824, NVP-LBH589, MS-275, PXD101, and FR901228. Compounds that inhibit expression of a deacetylase may be RNAi of HDAC7 or siRNA of HDAC7. Such agents, if used in combination, may be formulated separately or in combination with other agents. In addition, methods may further comprise the use of one or more immunosuppressants that do not activate the T suppressor cells by increasing the acetylation level and/or protein level of FOXP3 selected from the group consisting of: corticosteroids, rapamycin, Azathioprine (Imuran), Mycophenolate (MFM or CellCept), Cyclosporine (Sandimmune), Mercaptopurine (6-MP), basiliximab, daclizumab, sirolimus, tacrolimus, Muromonab-CD3, cyclophosphamide, and methotrexate. Such agents, if used in combination, may be formulated separately or in combination with other agents. In some embodiments, a universal T cell suppressor stimulus (for example in protein or DNA form) and/or an antigen that cross reacts with an inflammatory response associated with said autoimmune disorder, coronary artery disease, allergy, allograft rejection, or sepsis/toxic shock, (for example in protein or DNA form) are administered in combination with GITL administration (for example in protein or DNA form) and further optionally in combination with one or more deacetylase inhibitors and/or compounds that inhibit expression of a deacetylase and/or one or more immunosuppressants and/or one or more acetyl transferase enhancers.

Pharmaceutical compositions may be formulated in conventional manner using one or more physiologically acceptable carriers, diluents, excipients or auxiliaries which facilitate processing of the active protein into preparations which can be used pharmaceutically. Proper formulation is dependent upon the route of administration chosen.

For topical administration the compounds of the invention may be formulated as solutions, gels, ointments, creams, suspensions; etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

Alternatively, the compounds may be in powder form for constitution with a suitable vehicle, e.g., sterile pyrogen-free water, before use.

The protein will generally be used in an amount effective to achieve the intended purpose. For use to treat or prevent inflammatory/autoimmune/transplantation rejection the protein is administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the 1050 as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

Some embodiments include to pharmaceutical compositions which comprise therapeutically effective amounts of one or more regulatory composition that activate the T suppressor cells by increasing the acetylation level and/or protein level of FOXP3. Some embodiments include to pharmaceutical compositions which comprise therapeutically effective amounts of one or more regulatory composition that activate the T suppressor cells by increasing the acetylation level and/or protein level of FOXP3 in combination with one or more universal T suppressor cell stimulus (for example in protein or DNA form) and/or one or more antigen(s) (for example in protein or DNA form) and/or one or more immunosuppressants that do not activate the T suppressor cells by increasing the acetylation level and/or protein level of FOXP3 and/or GITL protein and/or DNA encoding GITL. Some embodiments include to pharmaceutical kits which comprise a package that contains more than one container where each container contains one or more components to be delivered in combination according to methods of the invention. The containers may be for example separate containers provided as or within a single package, a single container or package with separate compartments such as a blister pack that has separate compartments.

### Drug Discovery Methods

The present invention provides methods of identifying compounds useful for treating an individual who has an autoimmune disorder or treating an individual who has an coronary artery disease or reducing the symptom of allergy of an individual, or reducing the risk of rejection of an allograft in an individual undergoing immunosuppression, or treating an individual who has or is at an elevated risk of getting sepsis/toxic shock and methods of identifying compounds useful for treating cancer, infectious diseases or immune deficiency.

The methods of identifying compounds useful for treating an individual who has ari autoimmune disorder or treating an individual who has an coronary artery disease or reducing the symptom of allergy of an individual, or reducing the risk of rejection of an allograftin an individual undergoing immunosuppression, or treating an individual who has or is at an elevated risk of getting sepsis/toxic shock comprise the steps of: performing an assay to determine if a test compound increases acetylation level and/or protein level of FOXP3 in a suppressor T cell; and performing an assay to determine if the test compound that increases acetylation level and/or protein level of FOXP3 in a suppressor T cell is active in an animal model useful to evaluate a compound for activity to treat autoimmune disorder or coronary artery disease or allergy, or reducing the risk of rejection of an allograft, or sepsis/toxic shock. Assays to determine if a test compound increases acetylation level and/or protein level of FOXP3 in a suppressor T cell can be performed on isolated cells or using cell free compositions. According to some embodiments, T suppressor cells are isolated from PBMCs and contacted with a test compound. Acetylation level and/or protein level of FOXP3 is measured and compared to acetylation level and/or protein level of FOXP3 in cells either treated with a positive control (i.e. a compound known to increase acetylation level and/or protein level of FOXP3) and/or a negative control (i.e. a compound known to have no effect on acetylation level and/or protein level of FOXP3) or compared to known acetylation level and/or protein level of FOXP3 in the absence of compound that increases acetylation level and/or protein level of FOXP3. According to some embodiments, cells are contacted with a test compound. Acetylation level and/or protein level of FOXP3 is measured and compared to acetylation level and/or protein level of FOXP3 in cells either treated with a positive control (i.e. a compound known to increase acetylation level and/or protein level of FOXP3) and/or a negative control (i.e. a compound known to have no effect on acetylation level and/or protein level of FOXP3) or compared to known acetylation level and/or protein level of FOXP3 in the absence of compound that increases acetylation level and/or protein level of FOXP3. According to some embodiments, isolated FOXP3 is combined with a deacetylase and an acetyl donor in the presence of a test compound and an increase in acetylation level of FOXP3 indicates the test compound is a deacetylase inhibitor. According to some embodiments, isolated FOXP3 is combined with an acetyl transferase and an acetyl donor in the presence of a test compound and an increase in acetylation level of FOXP3 indicates the test compound is an acetyl transferase enhancer. Acetylation level of FOXP3 is measured and compared to acetylation level of FOXP3 in the presence of a positive control (i.e. a compound known to increase acetylation level and/or protein level of FOXP3) and/or a negative control (i.e. a compound known to have no effect on acetylation level and/or protein level of FOXP3) or compared to known acetylation level of FOXP3 in the absence of compound that increases acetylation level of FOXP3. In some embodiments, the deacetylase is HDAC. In some embodiments, the deacetylase is HDAC7. In some embodiments, the acetyl transferase is HAT. In some embodiments, the acetyl transferase is TIP60. Animal model useful to evaluate a compound for activity to treat autoimmune disorder or coronary artery disease or allergy, or reducing the risk of rejection of an allograft, or sepsis/toxic shock include the collagen induced arthritis model, the non-obese diabetic mouse model, the experimental autoimmune encephalomyelitis, and the Staph TSS model.. The steps may be performed in either order. In some preferred embodiments, the first step is screening of test compounds using assays to measure FOXP3 acetylation levels or protein levels and test compound found to be positive in the assay are subsequently tested in animal models.

The methods of identifying compounds useful for treating cancer, infectious diseases or immune deficiency comprising the steps of performing an assay to determine if a test compound decreases acetylation level and/or proteinievel of FOXP3 in a suppressor T cell; and performing an assay to determine if the test compound that decreases acetylation level and/or protein level of FOXP3 in a suppressor T cell is active in an animal model useful to evaluate a compound for activity to treat cancer, infectious diseases or immune deficiency. Assays to determine if a test compound decreases acetylation level and/or protein level of FOXP3 in a suppressor T cell can be performed on isolated cells or using cell free compositions. According to some embodiments, T suppressor cells are isolated from PBMCs and contacted with a test compound. Acetylation level and/or protein level of FOXP3 is measured and compared to acetylation level and/or protein level of FOXP3 in cells either treated with a positive control (i.e. a compound known to decrease acetylation level and/or protein level of FOXP3) and/or a negative control (i.e. a compound known to have no effect on acetylation level and/or protein level of FOXP3) or compared to known acetylation level and/or protein level of FOXP3 in the absence of compound that increases acetylation level and/or protein level of FOXP3. According to some embodiments, cells are contacted with a test compound. Acetylation level and/or protein level of FOXP3 is measured and compared to acetylation level and/or protein level of FOXP3 in cells either treated with a positive control (i.e. a compound known to decrease acetylation level and/or protein level of FOXP3) and/or a negative control (i.e. .a compound known to have no effect on acetylation level and/or protein level of FOXP3) or compared to known acetylation level and/or protein level of FOXP3 in the absence of compound that increases acetylation level and/or protein level of FOXP3. According to some embodiments, isolated FOXP3, is combined with an acetyl transferase and an acetyl donor in the presence of a test compound and a decrease in FOXP3 acetylation levels indicates the test compound is an acetyl transferase inhibitor. According to some embodiments, isolated FOXP3, is combined with a deacetylase and an acetyl donor in the presence of a test compound and a decrease in FOXP3 acetylation levels indicates the test compound is a deacetylase enhancer. Acetylation level of FOXP3 is measured and compared to acetylation level of FOXP3 in the presence of a positive control (i.e. a compound known to decrease acetylation level and/or protein level of FOXP3) and/or a negative control (i.e. a compound known to have no effect on acetylation level and/or protein level of FOXP3) or compared to known acetylation level and/or protein level of FOXP3 in the absence of compound that increases acetylation level and/or protein level of FOXP3. In some embodiments, the acetyl transferase is HAT. In some embodiments, the acetyl transferase is TIP60. In some embodiments, the deacetylase is HDAC. In some embodiments, the deacetylase is HDAC7. Animal model useful to evaluate a compound for activity to treat treating cancer, infectious diseases or immune deficiency are well know and include transgenic oncomice, influenza model, and herpes model. The steps may be performed in either order. In some preferred embodiments, the first step is screening of test compounds using assays to measure FOXP3 acetylation levels or protein levels and test compound found to be positive in the assay are subsequently tested in animal models.

### Examples

### Example 1

FOXP3 associates with TIP60 and HDAC7. Our initial studies documented that FOXP3 associated with TIP60. FOXP3 also exists in cytoplasmic and nuclear sites and its expression is linked to the suppressor phenotype in some cells. Because of the complexity of HAT and HDAC ensemble formation we examined interactions of FOXP3 with both Tip 60 and HDAC7. We used 293 T cells which were transfected with expression plasmids for human FOXP3, Flag-tagged TIP60, and Flag tagged HDAC7. The cells were lysed 48 hours later and then subjected to immunoprecipitation with anti-Flag M2 mAb. After transfer, western blots were performed with a variety of antibodies.

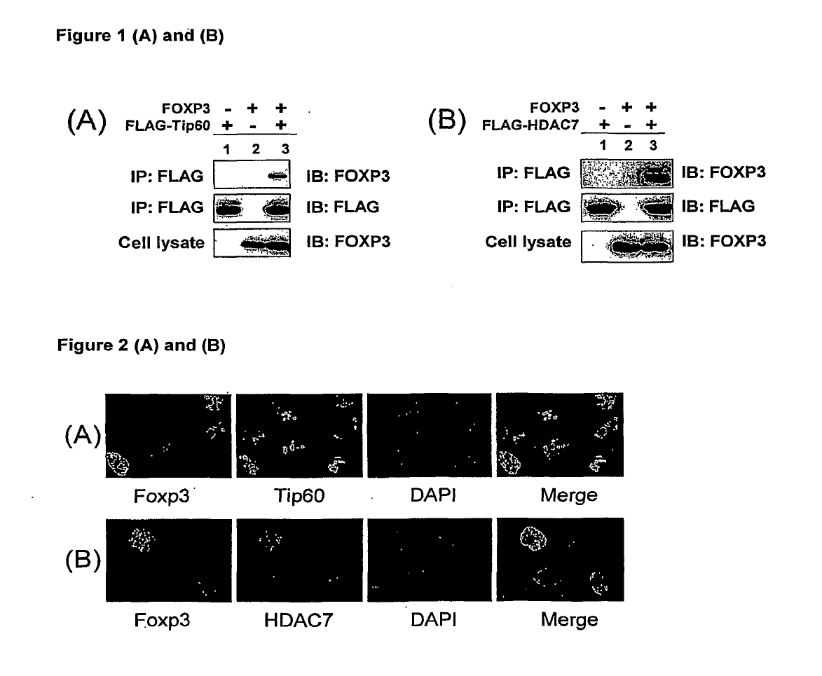

In these preliminary studies using transient expression, we found that both HDAC7 and TIP60 associated with FOXP3. As shown in FIGS. 1(A) and (B), FOXP3 associates with histone acetyltransferase TIP60 and histone deacetylase HDAC7 in vivo. FIG. 1(A) shows HEK 293T cells transfected with expression plasmids for human FOXP3 and, FLAG-tagged TIP60. FIG. 1(B) shows HEK 293T cells transfected with expression plasmids for human FOXP3 and FLAG-tagged HDAC7. 48 hours later RIPA buffer cell lysates were immunoprecipitated (IP) with anti-FLAG M2 mAb, then analyzed by Western blotting with indicated antibodies (IB).

FOXP3, TIP60 and HDAC7 all colocalize to nuclear sites. To extend these observations using ectopically expressed proteins, we examined U2OS cells transfected with GFP tagged human FOXP3, Flag tagged TIP60 and Flag tagged HDAC7. 48 hours after transfection the cells were stained with anti-Flag M2 mAb and detected with Texas red conjugated anti-mouse mAb. The localization of FOXP3, TIP60 and HDAC7 was addressed using conventional fluorescence microscopy. DAP1 was used to identify the nucleus.

Merge analysis revealed that FOXP3, TIP60 and HDAC7 were clearly associated in nuclear sites. FIG. 2, (A) and (B) show co-localization of FOXP3 with TIP60 and HDAC7 in the cell nucleus. U2OS cells were transfected with expression plasmids for GFP-tagged human FOXP3 and, FLAG-tagged TIP60 (FIG. 2(A)), or FLAG-tagged HDAC7 (FIG. 2(B)). 48 hours later cells were immunostained with anti-FLAG M2 mAb and detected with Texas Red conjugated anti-mouse mAb. FOXP3 (green) and TIP60 (red) or HDAC7 (red) subcellular localization were examined under conventional fluorescence microscopy. The blue channel shows the cell nucleus staining with DAPI. The specific molecular interactions among FOXP3, TIP60 and HDAC7 remain to be determined as do the endogenous associations of these proteins.

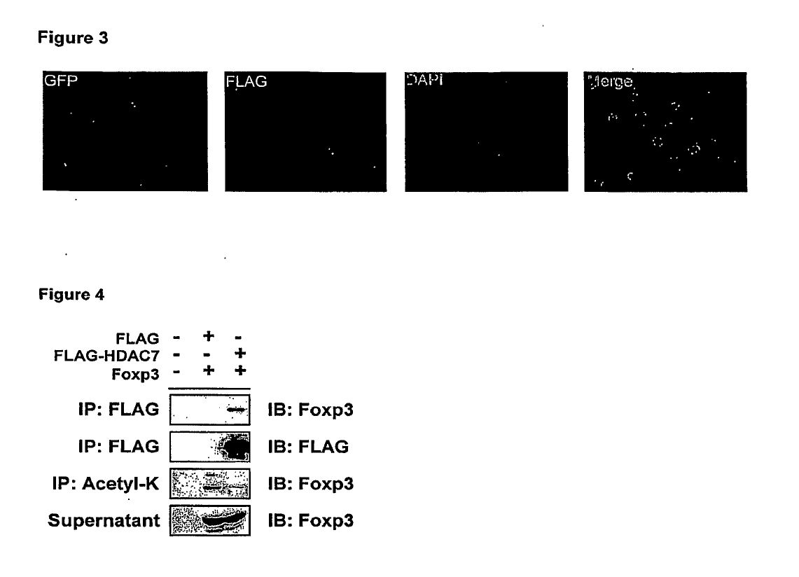

Overexpression of TIP60 promotes the nuclear accumulation of FOXP3. FIG. 3 shows data from Hela cells stably expressing GFP-FOXP3 that were transfected with pCMV-FLAG-tagged TIP60 expression vector. 24 hours later cells were fixed, then immunostained with anti-FLAG M2 mAb, and detected with Cy5 conjugated anti-mouse mAb (red). Cell nucleus is stained with DAPI as blue color.

Acetylation of FOXP3 and its interaction with HDAC7 is shown in FIG. 4. FOXP3 associates with histone deacetylase HDAC7 in vivo. HEK 293T cells were transfected with expression plasmids for human FOXP3 and FLAG-tagged HDAC7. 48 hours later, RIPA buffer cell lysates were immunoprecipitated (IP) with anti-FLAG M2 mAb, then analyzed by western blotting with indicated antibodies (IB).

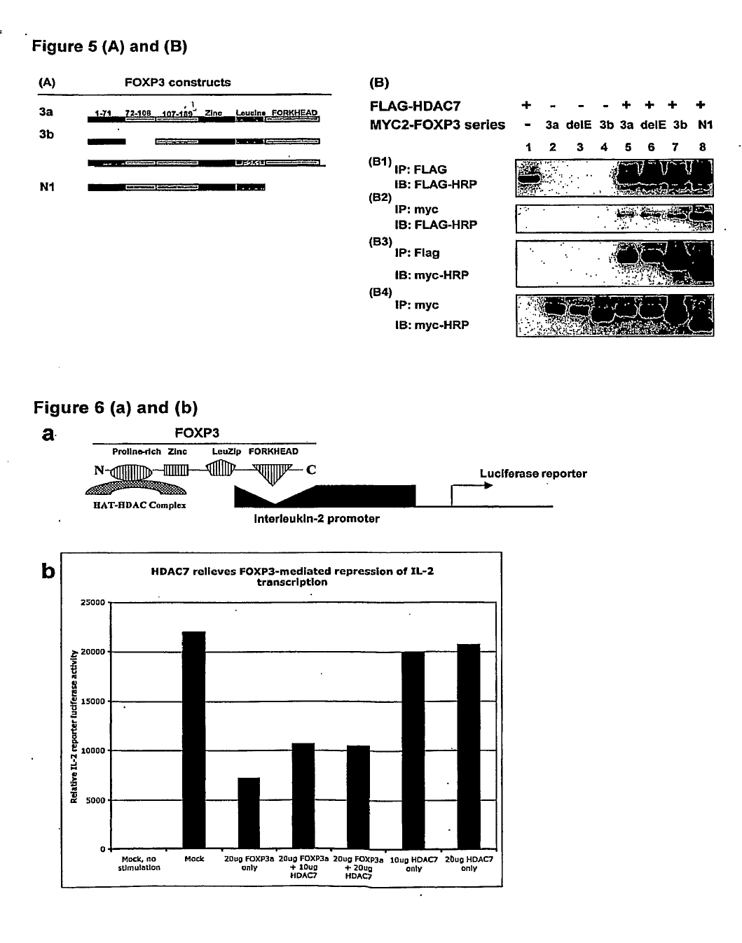

The N-terminal FOXP3 associates with HDAC7. FIG. 5(A) shows a schematic representation of FOXP3 series constructs used in cotransfection in HEK293T cells. FIG. 5(B) shows data from HEK 293T cells that were transfected with expression plasmids for FLAG-tagged HDAC7 and various myc-tagged FOXP3 vectors. 48 hours later, RIPA buffer cell lysates were immunoprecipitated (1P) with anti-FLAG M2 mAb, then analyzed by western blotting with indicated antibodies (IB).

HDAC7 relieves FOXP3-mediated suppression on IL-2 promoter. FIG. 6(a) shows a schematic representation of the FORKHEAD domain of FOXP3 binding to the human IL-2 promoter luciferase reporter. FIG. 6(b) shows data from Jurkat E6.1 T cells that were transfected with the control empty vector (mock), or vectors expressing wild type FOXP3 alone, FOXP3 with different amounts of HDAC7 together, or HDAC7 alone, plus full length IL-2-Luciferase reporter and control TK-Renilla luciferase vector as indicated. 24 hours after the electroporation, cells were stimulated with 50 ng/ml of PMA and 1 μM ionomycin for 6 hours before lysing and analyzed by means of dual luciferase assay normalized with Renilla luciferase activity. Results presented are means of 3 separate experiment's with SD.

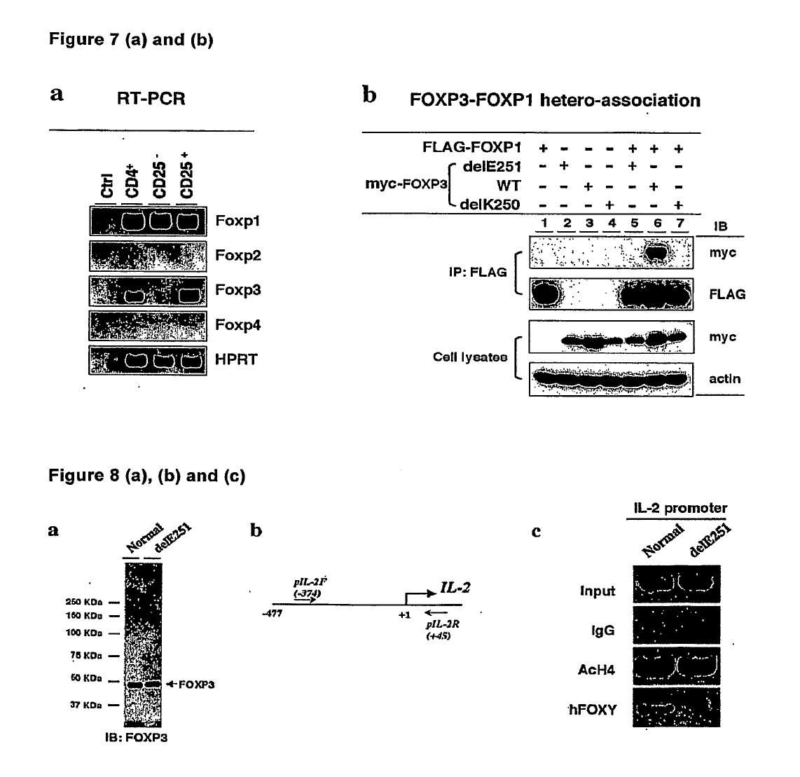

Wild type FOXP3, but neither K250 nor E251 deletion mutants found in human XLAAD patients, hetero-associate with its subfamily member FOXP1. FIG. 7 (a) shows Foxp1, Foxp2, FOXP3 and Foxp4 transcription levels in CD4+CD25+ and CD4+CD25− T cells. 99% pure populations of CD4+, CD4+CD25+ and CD4+CD25− T cells from CBA/Ca splenic T cells were isolated using a MACS CD4+ purification kit and further sorted into CD25+or CD25− subpopulations by FACStar PLUS cell sorter. cDNA from each population was subjected to nonsaturating PCR using specific primer pairs to Foxp1, Foxp2, FOXP3, Foxp4 or HPRT. The control lanes contain no template. FIG. 7(b) shows FOXP3 heterodimerizes with FOXP1 in vivo. HEK 293T cells were cotransfected with expression plasmids for FLAG-tagged FOXP1 (FLAG-FOXP1), myc-tagged wild-type FOXP3 (WT), FOXP3 E251 deletion mutant (delE251) and FOXP3 K250 deletion mutant (delK250). Forty-eight hours post transfection cell lysates were immunoprecipitated with anti-FLAG M2 mAb, then analyzed by Western blotting with anti-myc-tag 4E10 mAb, or anti-FLAG M2 mAb. FOXP3 and actin expression levels in cell lysates were analyzed with anti-myc or anti-actin antibodies.

Endogenous wild type FOXP3, but not the FOXP3 E251 deletion mutant, associates with the human IL-2 promoter in activated T cells. FIG. 8(a) shows nuclear extracts from primary normal and XLAAD patient (delE251) T cell lines that were immunoblotted with anti-human FOXP3 monoclonal antibody 221D. FIG. 8(b) shows a schematic representation of the primers used for detection on human IL-2 promoter region. FIG. 8(c) shows chromatin immunoprecipitation results showing that wild type FOXP3 from normal activated T cells but not the mutant FOXP3 from XLAAD patient T cells (delE251) associates with IL-2 promoter. Input, DNA from 1% of input for immunoprecipitation as positive control; IgG, normal mouse IgG; AcH4, anti-acetyl-histone 4 antibody as the positive control for transcriptionally active chromatin; hFOXY, a mouse anti-human FOXP3 monoclonal antibody which could equally immunoprecipitate endogenous wild type FOXP3 and E251 mutated FOXP3 proteins from total cell lysates (not shown). We performed a double immunoprecipitation process for each antibody to minimize antibody nonspecific binding.

### Example 2

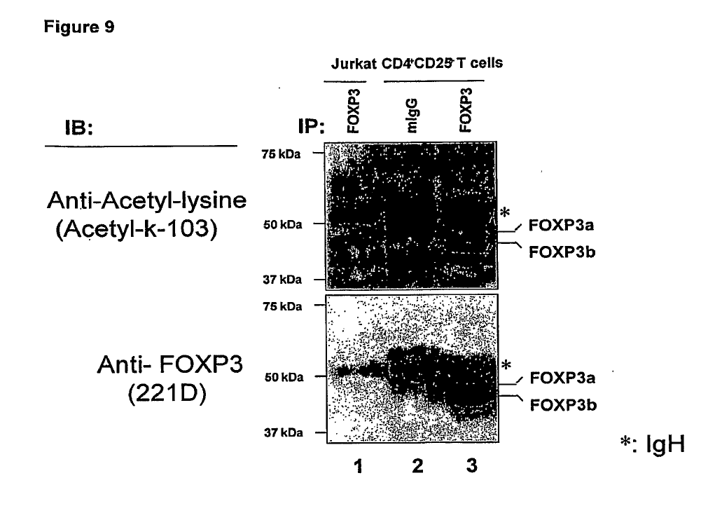

Endogenous FOXP3 acetylation in human CD4+CD25+ T cells: To verify whether endogenous FOXP3 is also acetylated under physiological conditions, comparable amounts of nuclear extracts were immunoprecipitated from human FOXP3 expressing CD4+CD25+ regulatory T cells or control Jurkat T cells that lack FOXP3 expression using either monoclonal anti-FOXP3 Ab hFOXY or control IgG, then immunoblotted with rabbit anti-acetyl-lysine polyclonal antibody (Upstate) (FIG. 9, upper panel). After stripping, the immunoblots were reprobed with anti-FOXP3 mAb 221D (FIG. 9, bottom panel). These studies confirmed that endogenous FOXP3 is acetylated in primary human CD4+CD25+ regulatory T cells expanded in vitro.

### Example 3

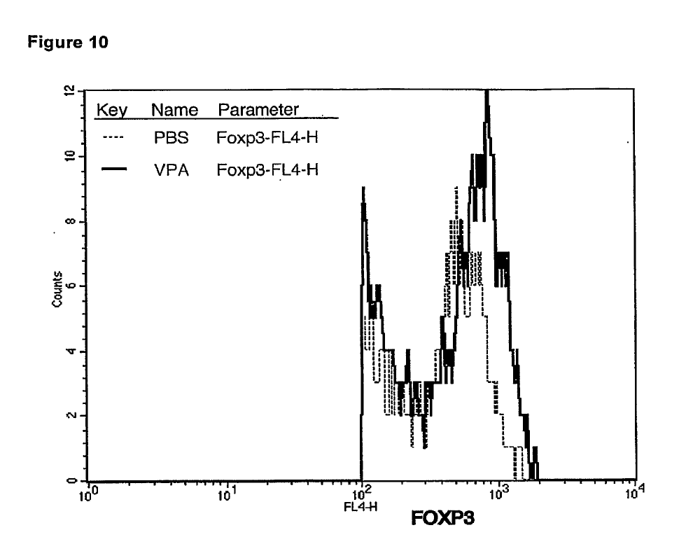

HDAC inhibitor treatment in collagen induced arthritis: HDAC inhibitor VPA was tested in the collagen induced arthritis (CIA) disease model of rheumatoid arthritis. CD4+ T cells as well as B cells are responsible for disease manifestation within the joints and CD25+ Treg play a protective role in disease development. After disease induction with type II collagen, mice were administered VPA (400 mg/kg) or PBS ip daily for 5 weeks. They were scored for severity of disease twice per week. In groups of 10-12 mice, 100% of mice receiving PBS showed signs of disease, whereas only 25% of mice receiving VPA showed any disease at the end of the 5 week study. Additionally, disease severity was greatly reduced in VPA treated mice compared to PBS treated. Splenocytes from collagen induced arthritic mice that were treated with VPA (no disease evident) or PBS (disease evident) were analyzed for expression of CD4, CD25 and FOXP3 by flow cytometry. A 1.63-fold increase in the percentage of FOXP3+CD25+ cells was observed in VPA treated versus PBS treated splenocytes. Additionally, the intracellular levels of FOXP3 protein were elevated in the VPA treated mice compared to PBS mice (FIG. 10). This analysis suggests that VPA treatment increases the number of FOXP3+CD25+ cells and also increases or stabilizes the FOXP3 protein in this population. In proposed studies VPA treated CD25+ cells will be compared to PBS treated CD25+ cells for increased functional regulatory activity in vitro. HDAC inhibitor VPA is able to modify disease progression in the CIA model of rheumatoid arthritis.

### Example 4

As shown by the evidence herein, FOXP3 actively represses transcription through its association with transcriptional corepressors histone acetyltransferase TIP60 and histone deacetylase HDAC7 and HDAC9 in vivo: The N-terminal 106-190aa proline-rich region of FOXP3, which has little similarity with other FOXP subfamily members, is the critical region for FOXP3 forkhead domain-mediated transcriptional repression, dependent on its dynamic association with TIP60 and HDAC7. Moreover, FOXP3 is acetylated in primary human Treg cells and this process is promoted by TIP60. While overexpression of TIP60, but not its HAT-deficient mutant, promotes FOXP3 mediated transcriptional repression, endogenous knockdown of TIP60 relieves this repression.

### Results

### FOXP3 is Acetylated, a Process Promoted by Histone Acetyltransferase TIP60

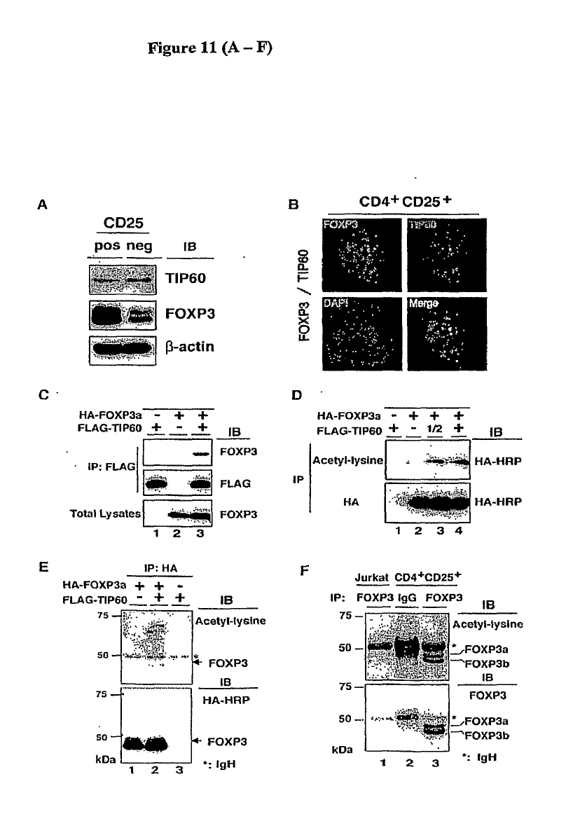

Histone acetyltransferase, the HIV-1 TAT-interactive protein, 60 kDa (TIP60), has been shown to associate with the C-terminal proline-rich domain of an adaptor protein Cas-Br-M (murine) ecotropic retroviral transforming sequence b (Cbl-b) by yeast two hybrid screening The N-terminal proline rich region of FOXP3 could also interact with TIP60. TIP60 expression in human CD4⁺CD25⁺ T cells and CD4⁺CD25⁻ T cell was studied by immunoblotting with rabbit anti-TIP60 polyclonal antibody (Upstate), and found that TIP60 was expressed almost equally in both cell types (FIG. 11A, upper panel). Interestingly, in vitro expanded CD4⁺CD25⁻ T cells also expressed a small but detectable amount of FOXP3 (FIG. 11A, middle panel). Endogenous TIP60 co-localizes with FOXP3 in the nucleus of human CD4⁺CD25⁺ regulatory T cells, results from co-immunostaining with mouse anti-human FOXP3 monoclonal antibody hFOXY (eBioscience), and rabbit anti-T1P60 (Upstate) are shown in FIG. 11B.

To determine whether FOXP3 associates with TIP60 in vivo, a hemagglutinin (HA)-tagged full length FOXP3 (FOXP3a) expression construct was co-transfected together with a FLAG tagged TIP60 expression construct into 293T cells. 48 hours after transfection, cell lysates in RIPA buffer were immunoprecipitated with anti-FLAG M2 antibody, followed by Western blotting with anti-FOXP3 antibody 221 D, and reprobed with anti-FLAG M2. Total cell lysates were also analyzed for FOXP3 expression. FOXP3 co-immunoprecipitated with TIP60 (FIG. 11C).

Because TIP60, as a histone acetyltransferase, acetylates non-histone transcription factors such as c-Myc, experiments were performed to determine whether FOXP3 is acetylated in vivo. The HA-tagged full-length FOXP3 (FOXP3a) expressing construct was co-transfected with increasing amounts of FLAG tagged TIP60 construct into 293T cells. Cell lysates in RIPA buffer were immunoprecipitated with anti-acetyl-lysine Ac-k-103, then analyzed with anti-HA-HRP (FIG. 11D). Overexpressing TIP60 permitted anti-acetyl-lysine mAb Ac-k-103 to immune precipitate increasing amounts of FOXP3 proteins, indicating TIP60 promotes FOXP3 acetylation (FIG. 11D).

Reciprocal precipitations were performed as well. HA-FOXP3a and FLAG-TIP60 transfected HEK293T cells were immunoprecipitated with anti-HA mAb F-7 (Santa Cruz), then immunoblotted with rabbit anti-acetyl-lysine polyclonal antibody (Upstate), and reprobed with anti-HA-HRP (Roche). As shown in FIG. 11E, overexpression of TIP60 promoted FOXP3 acetylation, as well as acetylation of several unknown proteins with molecular weights ranging between 50 kDa and 75 kDa that all exist in the FOXP3 immunoprecipitated complex (left panel, upper bands).

Furthermore, to verify whether endogenous FOXP3 is also acetylated under physiological conditions, comparable amounts of nuclear extracts from human FOXP3 expressing CD4⁺CD25⁺ regulatory T cells or control Jurkat T cells that lack FOXP3 expression were immunoprecipitated with either monoclonal anti-FOXP3 Ab hFOXY or control IgG, then immunoblotted with rabbit anti-acetyl-lysine polyclonal antibody (Upstate) (FIG. 11F, upper panel). After stripping, we reprobed with anti-FOXP3 mAb 221D (FIG. 11F, bottom panel). These studies confirmed that endogenous FOXP3 is acetylated in primary human CD4⁺CD25³⁰ regulatory T cells expanded in vitro.

FOXP3 Associates with Class II Histone Deacetylase HDAC7 in Human CD4⁺CD25⁺ T Cells

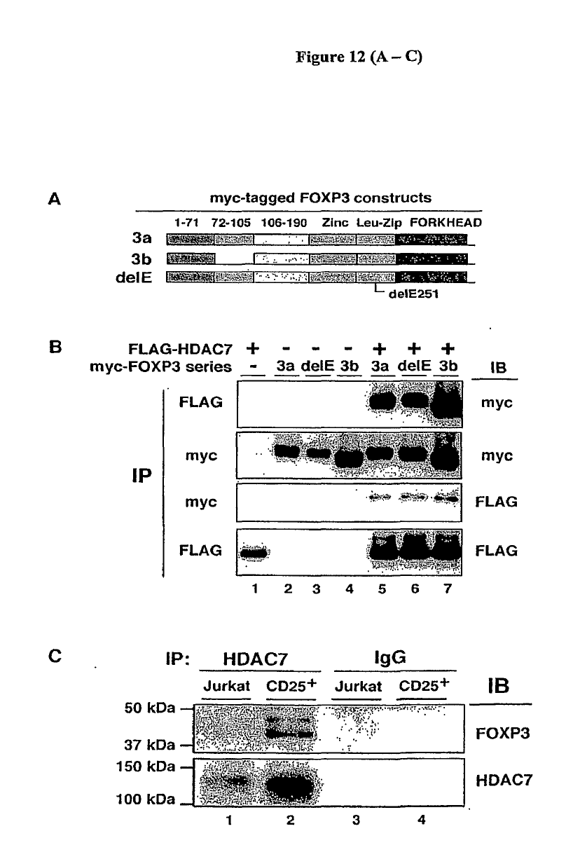

Because the histone acetyltransferase TIP60 associates with and recruits histone deacetylase HDAC7 for transcriptional repression in other repressor complexes, experiments were performed to determine whether FOXP3 also existed in association with histone deacetylase HDAC7. Interactions of FOXP3 with HDAC7 were examined using full length FOXP3a and the exon 2 lacking isoform FOXP3b, as well as the human IPEX patient delE251 mutant FOXP3 (FIG. 12A). A panel of myc-tagged FOXP3 expression constructs were co-transfected a together with FLAG tagged HDAC7 expression constructs into 293T cells (FIG. 12A). The cells were lysed 48 hours later and then subjected to immunoprecipitation with anti-FLAG M2 mAb or anti-myc mAb 9E10, followed by western blotting with anti-myc mAb or anti-FLAG M2 mAb. HDAC7 associated with both the large and small isoform of FOXP3 (FIG. 12B, lane 5 and lane 7). This association is clearly independent of the ability of FOXP3 to undergo dimerization or tetramerization since it is not affected by the IPEX patient mutant delE251 that we have shown exists as a monomer and cannot form higher ordered species (FIG. 12B, lane 6).

It was then determined that FOXP3 associates with HDAC7 under physiologic conditions. Nuclear extracts from FOXP3 expressing primary human CD4⁺CD25⁺ regulatory T cells or an equal number of control Jurkat T cells that lack FOXP3 expression were immune precipitated with either goat anti-HDAC7 (C-18) or control IgG, then immunoblotted with anti-FOXP3 mAb 221D. After stripping, we then reprobed with rabbit anti-HDAC7 (KG-17). These studies established the endogenous FOXP3 association with HDAC7 in in vitro expanded primary human CD4⁺CD25⁺ regulatory T cells (FIG. 12C).

### The N-Terminal 106-190 aa Region of FOXP3 as a Transcriptional Repression Domain

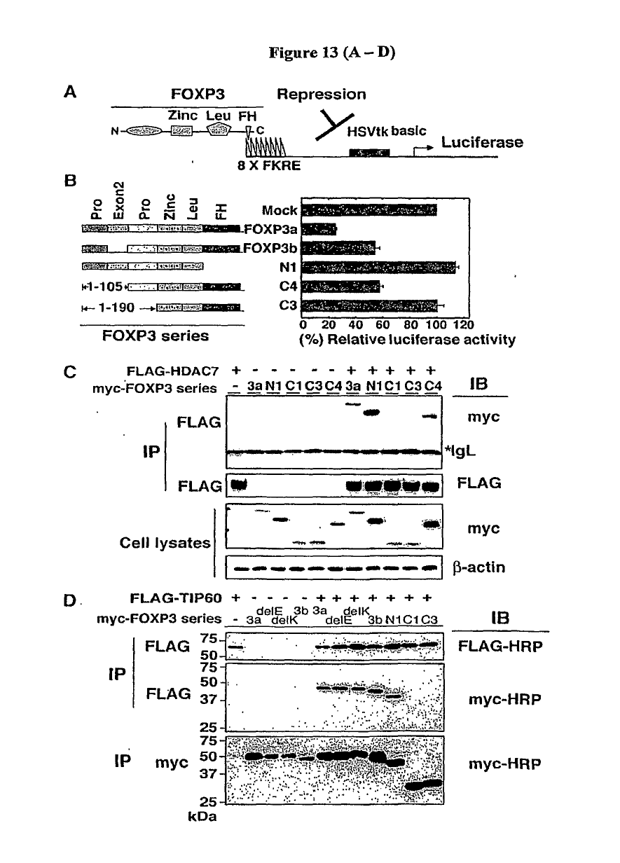

FOXP3 contains an N-terminal proline-rich region, a C2H2 zinc finger domain, a leucine zipper domain and a C-terminal forkhead domain. To determine which subdomains of FOXP3 are responsible for its transcription repressive activity, a panel of FOXP3 expression constructs were co-transfected together with a firefly luciferase reporter 8×FK1tk-Luc construct driven by a specific upstream promoter/enhancer with 8 forkhead binding sites in 293T cells (FIG. 13A). The large isoform of FOXP3 (FOXP3a) was found to have the maximal transcription repression activity, while the forkhead deletion mutation of FOXP3 (N1) has completely lost its repressive activity as expected. The smaller isoform of FOXP3 lacking exon 2 (FOXP3b) still mediated repression, as did the N-terminal 1-105 aa deletion mutant C4. However, the N-terminal 1-190aa deletion mutant (C3) with the intact forkhead DNA binding domain was unable to repress transcription from the reporter (FIG. 13B). These data clearly show that the N-Terminal region between amino acids 106-190 is essential.and critical to FOXP3 mediated transcriptional repression induced by forkhead domain binding to DNA.

Since FOXP3 associates with HDAC7 and TIP60, which also interact with each other and have been suggested to act as transcriptional repressors, experiments were performed to test whether the N-Terminal 106-190aa transcription repression domain was also essential for the association of FOXP3 with HDAC7 and TIP60. A series of myc-tagged FOXP3 expression constructs including the full length FOXP3a (1-431 aa), the FOXP3 forkhead deletion mutant (N1), the N-terminal 1-220 aa deletion mutant (C1), the N-terminal 1-190aa deletion (C3), and N-terminal 1-105aa deletion mutant (C4) together with the FLAG-tagged HDAC7 expression construct were transfected into 293T cells. 48 hours later cell lysates were immunoprecipitated with anti-FLAG M2 mAb, then immunoblotted with either anti-myc 9E10 mAb to detect FOXP3 association, or reprobed with anti-FLAG M2 to confirm immunoprecipitation of HDAC7. Total cell lysates were immunoblotted with either anti-myc 9E10 to show FOXP3 expression level or anti-beta actin antibody as loading control (FIG. 13C). Although the FOXP3 forkhead deletion mutant (NI), and N-terminal 1-105aa deletion mutant (C4) could still associate with HDAC7, the N-terminal 1-190aa deletion (C3) or N-terminal 1-220 aa deletion (C1), limited FOXP3's association with HDAC7.

Similar experiments were performed to map the subdomain of FOXP3 associated with TIP60 which interacts with HDAC7. TIP60 associated with the large isoform FOXP3a, the small isoform FOXP3b (with the 71-105aa deletion), de1E251 FOXP3a, delK250 FOXP3a and FORKHEAD deleted FOXP3 (N1) (FIG. 13D), and N-terminal 1-105aa deletion mutant (C4), but not the N-terminal 1-220 aa deletion (C1) or N-terminal 1-190aa deletion of FOXP3 (C3) (FIG. 13D). Together, the coincidence of N-terminal 106-190 aa as an essential region for FOXP3 transcription repression activity, as well as the importance of this region for association with transcription co-repressors HDAC7 and TIP60, suggests one molecular mechanism by which a tripartite ensemble of TIP60, HDAC7 and FOXP3 functions as a transcriptional repressor in vivo.

Knockdown of Endogenous TIP60 Relieves FOXP3 Mediated Repression In vivo

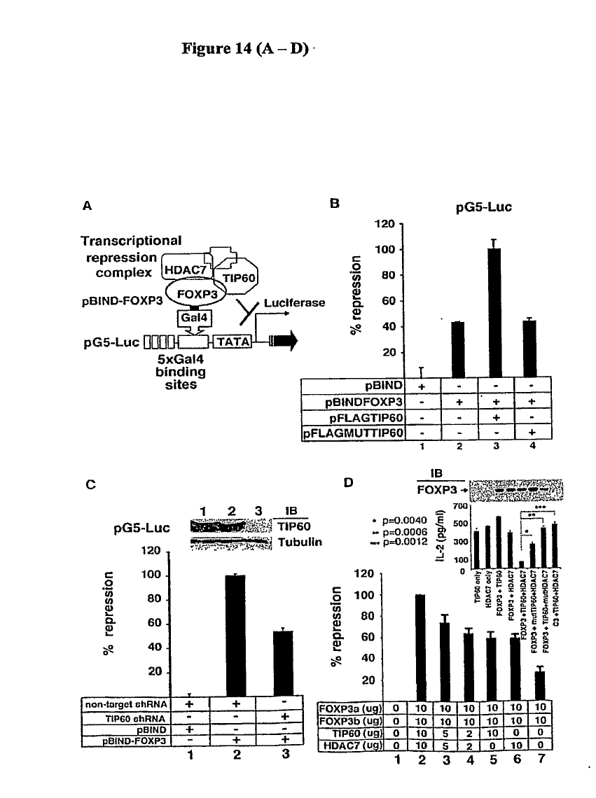

To evaluate whether the molecular mechanism of FOXP3 mediated repression is dependent on TIP60, a transcriptional repression assay (illustrated in FIG. 14A) was established. Ga14-FOXP3a fusion protein expressing vector pBIND-FOXP3a, the five Ga14-binding site driven firefly luciferase transcription reporter pGSluc (Promega), pMSVβgal control vector, were cotransfected together with pFLAG vectors expressing TIP60 or HAT-deficient TIP60(pFLAGMUTTIP60) in 293T cells. Full-length FOXP3a, when expressed as a Ga14 fusion protein, acted as a repressor of the five Gal4-binding site driven firefly luciferase transcription reporter (FIG. 14B, lane 2). Over expression of wild type TIP60 (FIG. 14B, lane 3), but not the HAT-deficient TIP60 (FIG. 14B, lane 4) promotes Gal4-FOXP3 mediated transcriptional repression. Therefore the HAT activity of TIP60 is important for this transcription repressive activity.

It was next established that the endogenous TIP60 expression in 293T cells contributes to FOXP3 mediated transcriptional repression. Experiments were performed to test whether knockdown of endogenous TIP60 expression would affect FOXP3 mediated repression. Inhibition of the endogenous TIP60 expression dramatically relieved FOXP3 mediated transcriptional repression (FIG. 14C, lane 3). The non-targeted shRNA construct (Sigma) was employed as a negative control (FIG. 14C, lane 2). Endogenous TIP60 knockdown was also performed by lentivirus based shRNA transduction into in vitro expanded human CD4⁺CD25⁺ regulatory T cells. The TIP60 shRNA transduction dramatically reduced proliferation and viability of the slowly expanding lentiviral transduced CD4⁺CD25⁺ regulatory T cells compared to the non-targeted shRNA transduced cells preventing appropriate biochemical analyses. This observation is consistent with the known early lethal effect of TIP60 knockout in mice. The knock down data of endogenous TIP60 support the conclusion that TIP60 is an essential subunit of the FOXP3 repression complex, and the HAT enzymatic activity of TIP60 plays an important role in repression mediated by the FOXP3 complex.

### A FOXP3 Ensemble is Necessary for IL-2 Production Regulation

FOXP3-TIP60-HDAC7 ensemble was examined to study its effects on IL-2 production. Coexpression of both FOXP3 isoforms was used to mimic the physiological expression pattern of human FOXP3 in vivo. Jurkat E6.1 T cells were cotransfected with both FOXP3a and FOXP3b, together with or without TIP60 alone, HDAC7 alone or the titrated amounts of the combination of both TIP60 and HDAC7. As depicted in FIG. 14D, the combination of TIP60 and HDAC7 with FOXP3a and FOXP3b led to the maximal repression in a dose dependent manner (lane 2,3,4), and the combination of either TIP60 or HDAC7 alone with FOXP3a and FOXP3b reduced the total repression efficiency (lane 5,6). FOXP3a and FOXP3b alone also repressed IL-2 production to a degree as a consequence of recruitment of endogenous HAT-HDAC complexes (FIG. 14D, lane 7). The inset in FIG. 14D shows the actual amount of IL-2 produced after transfections with different forms of Tip60 or HDAC7. Clearly optimal suppression of IL2 production by Foxp3 requires both intact enzymes.

Therefore, data suggest that FOXP3 recruitment of a functional HAT/HDAC complex is essential for its repression of cytokine production and that expression of wild type HATs or HDACs can directly affect the stoichiometric and functional features of the ensembles needed for regulation.

TCR Stimulation Disrupts FOXP3 and HDAC9 Interaction, which can be Restored by TSA Treatment

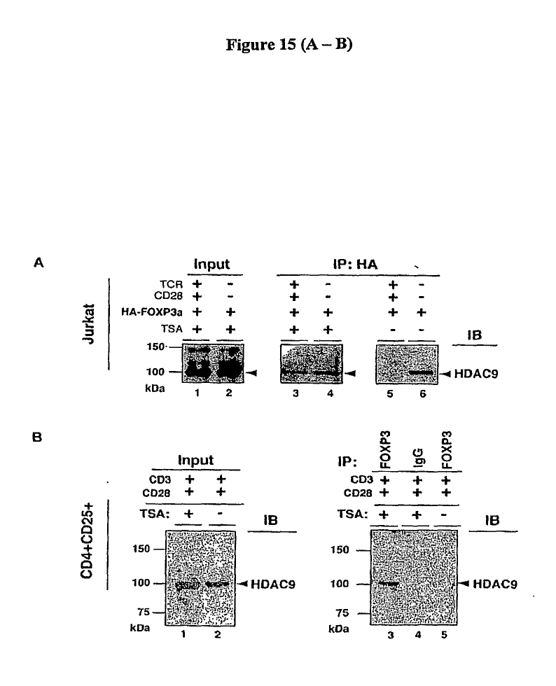

Experiments were performed to test whether other class II histone deacetylases expressed in T cells, such as HDAC9, exist in the FOXP3 complex under different physiological conditions. HDAC9 expression is enriched in naive Foxp3⁺ Treg cells. HA-tagged FOXP3a expressing Jurkat T cells were stimulated with plate bound anti-TCR V□ 8.1 plus soluble anti-CD28 for 4 hours, in the presence or absence of 400 nM of the histone deacetylase inhibitor TSA (FIG. 15A). The FOXP3 complex was immunoprecipitated from the nuclear extracts with the anti-HA probe F7, then immunoblotted with rabbit anti-HDAC9 (H-45) antibody that specifically recognizes the N-terminal 1-45 aa of human HDAC9 isoforms 1-4 (Santa Cruz). In the absence of T cell stimulation (FIG. 15A, lane 4, 6) HDAC9 co-immunoprecipitates with FOXP3. However, TCR plus CD28 stimulation is sufficient to antagonize the FOXP3 complex from its association with endogenous HDAC9 (FIG. 15A, comparing lane 5 and lane 6). Interestingly, the disruption of the FOXP3-HDAC9 complex can be reversed by treating these activated T cells with the HDAC inhibitor trichostatin A (TSA) (FIG. 15A, lane 3, 4). Endogenous FOXP3-HDAC7 exists in in vitro anti-CD3/CD28 activated and expanded human

CD4⁺CD25⁺ T cells (FIG. 12C). To confirm that protein HDAC function plays a role in the dynamic assembly of endogenous FOXP3-HDAC9 complex, we immunoprecipitated endogenous FOXP3 complex from in vitro anti-CD3/CD28 activated and expanded human CD4⁺CD25⁺ T cells, with or without pretreatment with TSA for 4 hours, followed by western blotting for HDAC9. Consistent with the result in Jurkat T cells, we also did not detect the endogenous association of FOXP3-HDAC9 in the in vitro anti-CD3/CD28 activated and expanded human CD4⁺CD25⁺ T cells (FIG. 15B, lane 5). However, TSA treatment is sufficient to promote endogenous FOXP3-HDAC9 association (FIG. 15B, lane 3). Together, these data indicate that in addition to a T cell receptor plus CD28 stimulation signal, HDAC activity may also play a determining role on the stability of the dynamic ensembles of the FOXP3-HDAC9 complex. The data suggest that HDAC9 subserves a different function than HDAC7 in FOXP3 mediated regulation and that disabling HDACs may alter regulatory T cell functions in vivo.

### Discussion

Specific recruitment of a histone acetyltransferase complex and histone deacetylase complex to target genes by transcription factors is relevant for transcriptional activation as well as transcriptional repression. FOXP3 exists as a large complex independent of FOXP3 oligomerization, which led to the exploration of the possible role of FOXP3 as a positive transcriptional repressor. The repressor function hypothesized might be further developed by recruitment of transcription co-repressors such as histone acetyltransferase and histone deacetylase. Data show histone acetyltransferase TIP60 and histone deacetylases HDAC7 and HDAC9 associated in a dynamic ensemble with FOXP3 in vivo. To date, TIP60 is the only detectable histone acetyltransferase coimmunoprecipitated with FOXP3 which suggests that TIP60 is the principal HAT responsible for FOXP3 acetylation and FOXP3-mediated transcriptional regulation in vivo. Moreover FOXP3 is acetylated and that this modification is linked to its function in T regulatory cells.

Titratable collaboration between TIP60 and HDAC7 appears important for FOXP3 mediated repression of IL-2 production in Jurkat E6.1 T cells after stimulation. Stoichiometric features of the FOXP3 ensembles may thus be crucial in the regulation of functional complex formation. Notably the N-terminal HAT/HDAC association domain deletion mutants of FOXP3 do not possess transcriptional repression activity even with an intact c-terminal Forkhead domain that mediates DNA interactions. The imbalance of overexpressed TIP60 or HDAC7, when transfected with FOXP3 alone, may affect other transcription factors. One transcription factor that might be affected is MEF2. MEF2 binds to the IL-2 promoter, and upregulates IL-2 production in T cells after stimulation. Multimeric ensembles have been found to suppress protein complex formation by disproportionately high concentrations of their individual components. Clearly there is a stoichiometric relationship of the constituents of the FOXP3-TIP60-HDAC7 complex that is needed to obtain the maximal repression of IL-2 production in T cells after TCR plus CD28 stimulation.

Histone acetyltransferases can directly interact with histone deacetylases in vivo, although the molecular mechanisms and consequences of such an arrangement are currently unclear. Notably TIP60 associates with HDAC7 through its N-terminal zinc finger-containing region and as supported by our studies that HDAC7 activity is required for certain of the repressive effects of TIP60.

The acetylation of histone H3 and H4 N-terminal tails is generally thought to increase accessibility of the transcriptional machinery to promote gene transcription. However, recent studies have indicated that HATs modify a variety of non-histone proteins. Most of these acetylated non-histone proteins include DNA-binding transcriptional factors, such as p53, BCL6, STAT3, FOXO4, the RelA subunit of NFicB. Consistent with its association with histone acetyltransferase TIP60, FOXP3 was found to be acetylated in primary Treg cells, and overexpression of TIP60 increased the acetylation level of FOXP3 as well as its associated proteins. Protein acetylation regulates transcription factor activity at multiple levels, including protein subcellular localization, DNA binding activity, and protein dimerization.

While endogenous FOXP3 consistently associated with HDAC7 in human in vitro activated and expanded regulatory T cells, FOXP3 was also found to be disassociated from another class II deacetylase, HDAC9, after TCR plus CD28 stimulation. The observation suggests a dynamic quality to FOXP3 complex ensembles that occurs in response to T cell receptor signals. Moreover, the dynamic association of FOXP3 and HDAC9 is promoted by the protein deacetylase inhibitor Trichostatin A. It is noteworthy that HDAC9 can function as a signal-responsive repressor independently of its HDAC catalytic domain.

Studies of the rational inactivation of HDACs have been initiated with the intent of modifying FOXP3 acetylation. Trichostatin A or VPA treatment has been used in several autoimmune models, and has shown significant clinical effects. This treatment leads to increased FoxP3⁺ suppressor cell levels and function. The models studied include the Nonobese Diabetic Mice (NOD) model, the myelin oligodendrocyte glycoprotein-induced experimental autoimmune encephalomyelitis (EAE) model, and the collagen-induced arthritis (CIA) mouse model.

At this point the totality of activities that TIP60 mediated FOXP3 acetylation effects has not been resolved. Which sites of FOXP3 that are acetylated also remain undefined. Based on observations that inhibition of endogenous TIP60 expression by shRNA knockdown relieved FOXP3 mediated transcriptional repression, the association of FOXP3 with HAT/HDACs may occur to facilitate the preferential transcription of FOXP3-targeted genes and serves as a mechanism whereby cellular repression is established and regulated. Moreover, the transcriptional repression effect of GAL4-FOXP3 is reduced but still detected even when the endogenous TIP60 expression had been completely knockdown by shRNA transduction. This finding indicates that other unknown transcriptional co-repressors could also contribute to FOXP3 mediated repression (FIG. 14C).

The in vitro activation of CD4⁺CD25⁺ T cell suppressor function also requires IL-2 function. FOXP3 has been shown to repress 1L-2 production by directly interacting with the conserved Forkhead binding sites near the NFAT site of the IL-2 promoter. Based on the biochemical analysis of FOXP3 complex in human CD4⁺CD25⁺ T cells, FOXP3 ensembles may be composed of multiple protein subunits, and the dynamic assembly of the FOXP3 complex may be responsible for the transcriptional repression of interleukin-2 in regulatory T cells. HAT-deficient TIP60 mutant mice may help to further clarify certain aspects of Tip60's physiological role in regulatory T cells in vivo.

FOXP3 can be acetylated in vivo and that overexpression of histone acetyltransferase TIP60 increases FOXP3 acetylation levels. The TIP60 and HDAC7 associated N-terminal 106-190aa of FOXP3 is required for FOXP3 mediated transcriptional repression and should be considered as a repression domain. Overexpression of the wild type TIP60, but not the HAT-deficient TIP60 promotes FOXP3 mediated repression, while knockdown of endogenous TIP60 relieved FOXP3 mediated repression. These studies suggest that stimuli signals which promote TIP60 function or decrease HDAC activity may modify T cell mediated suppression. Finally, these studies extend our notion that modifications by distinct acetyltransferases and deacetylases may occur on the same transcription factor perhaps at distinct sites, times, and after different signaling events.

The dynamic ensembles of FOXP3 with HAT/HDAC complexes provides a molecular explanation of how FOXP3 mediates transcriptional repression in regulatory T cells and identifies pharmaceutical approaches such as altering the enzymatic activity of HATs or HDACs to modify regulatory T cell functions.

### Experimental Procedures

### Human CD4⁺CD25⁺ T Cells

Human FOXP3⁺CD4⁺CD25⁺ T cells were obtained by in vitro expansion as follows: two hundred million PBLs were stained for CD4 and CD25, and using a Mo Flo high speed sorter, the brightest (top 1%) CD4⁺CD25⁺ cells were purified. These cells were stimulated with anti-CD3, anti-CD28 coated beads using a 3 bead to 1 cell ratio or using a cell based αAPC expressing CD64 and CD86 loading with anti-CD3 Ab in the presence of high levels of IL-2 (3000 U/ml) and cultured in RPMI with 10% FCS for the next 20-25 days.

Cloning of Human FOXP3 cDNA

Total RNA was isolated from 5 million expanded human FOXP3⁺CD4⁺CD25⁺ T cells. Based on the nucleotide sequence of FOXP3 in the human genome (http://www.ensembl.org/Homo_sapiens/), the following two primers we designed: 5′-CAAGGATCCGTATGCCCAACCCCAGG-3′ (SEQ ID NO:2) and 5′-ACAGTCTAGATCAGGGGCCAGGTGTAGGG-3′ (SEQ ID NO:3), to amplify FOXP3 cDNA with KOD Hot Start DNA Polymerase (Cat#71086, Novagen, Inc). Two PCR amplicons were separately digested with BamHI and XbaI and cloned into the mammalian expressing vectors pIRESpuromycin-myc2 with two myc epitope tags at the N-terminus or pIRESpuromycin-HA2 with two HA epitope tags, or pIRESpuromycin-FLAG2 with two FLAG epitope tags. DNA sequencing results showed that we cloned two FOXP3 cDNAs, one corresponding to the full length, which was used in all the following experiments as well as a splice variant lacking exon 2.

### Preparation of Nuclear Extracts

Nuclear extracts were prepared as follows. 20 million Human FOXP3⁺CD4⁺CD25⁺ T cells were washed with ice-cold DPBS and were incubated in hypotonic buffer containing 10 mM HEPES, pH7.9, 10 mM KCl, 0.1 mM EDTA, 1 mM DTT, 0.5 mM PMSF, 1× complete protease inhibitor cocktails (Cat. No. 1-697-498, Roche Biochem.), 1 mM Na₃VO₄ on ice for 15 min. Cells were then lysed in the presence of 0.6% NP-40 on ice for 15 min with vortexing. The nuclei were collected by centrifugation for 30 sec, and were resuspended in 100 ul of 20 mM HEPES p1-17.9, 400 mM NaCl, 1 mM EDTA, 1 mM DTT, 1 mM PMSF, 1× protease inhibitor cocktails, 1 mM Na₃VO₄, followed by rotation for 30 min at 4° C. The nuclear suspension was centrifuged at 13000 rpm, 4° C. for 15 min. The nuclear protein containing supernatant concentration was measured with BCATM Protein Assay Kit (Pierce) using bovine serum albumin as a standard.

### Plasmids, Reagents and Antibodies

The following antibodies were used: Affinity Purified anti-human FOXP3 monoclonal antibody hFOXY from eBioscience; anti-myc (9E10); HRP conjugated 9E10 and anti-HA-probe (F-7), anti-HDAC9 (H-45), anti-HDAC7 (C-18) from Santa Cruz Biotechnology; anti-FLAG M2; rabbit anti-HDAC7 (KG-17), anti-beta actin (AC-15), affinity-purified nonspecific IgG from Sigma; anti-TIP60 (07-038) from Upstate; anti-FOXP3 monoclonal Antibody 221D; HRP conjugated anti-HA 3F10 from Roche; HRP conjugated Rat anti-mouse kappa chain (04-6620) from Zymed; Cy3-conjugated anti-rabbit and FITC-conjugated anti-mouse IgG from Jackson ImmunoResearch Laboratory. Histone deacetylation inhibitor Trichostatin A (T 8552) was from Sigma. pcDNA-FLAG-HDAC-deficient HDAC7(H669F/H670F1H709F/H710F) construct was made by site-mutant kit. N-terminal Myc-tagged, HA-tagged, and FLAG-tagged series FOXP3 constructs 3a, N1, C1, C2, C3, C4 was made by PCR using full length FOXP3a as template except 3b using FOXP3b as template, then subcloned to the BamHI-XhoI sites of pIRESpuromycin-myc2, -HA2, -FLAG2 tagged empty vectors. The primers are the following:
3a (1-431aa) and 3b (with 71-105aa deletion):

(SEQ ID NO: 4)

5′-aag gat cca tgc cca acc cca ggc ctg-3′
and

(SEQ ID NO: 5)

5′-aat ctc gag tca ggg gcc agg tgt agg g-3′;
N1 (1-336aa):

(SEQ ID NO: 6)

5′-aag gat cca tgc cca acc cca ggc ctg-3′
and

(SEQ ID NO: 7)

5′-tac tcg agc atg ttg tgg aac ttg aag-3′;
C1 (221-431aa):

(SEQ ID NO: 8)

5′-gcg gat ccg acc atc ttc tgg atg ag-3′
and

(SEQ ID NO: 9)

5′-aat ctc gag cat gtt gtg gaa c-3′;
C3 (191aa-431aa):

(SEQ ID NO: 10)

5′-agg gat cct acc cac tgc tgg caa atg g-3′
and

(SEQ ID NO: 11)

5′-aat ctc gag tca ggg gcc agg tgt agg g-3′;
C4 (106aa-431aa):

(SEQ ID NO: 12)

5′tgg gat ccc tct caa cgg tgg-3′
and

(SEQ ID NO: 13)

5′-aat ctc gag tca ggg gcc agg tgt agg g-3′.

### Site-Directed Mutagenesis

The following primers were used to make FOXP3 delK250 and delE251 mutants respectively:

(SEQ ID NO: 14)

5′-gct ggt gct gga gga gaa gct gag tgc c-3′
and

(SEQ ID NO: 15)

5′-ggc act cag ctt ctc ctc cag cac cag c-3′;

(SEQ ID NO: 16)

5′-ctg gtg ctg gag aag aag ctg agt gcc atg-3′
and

(SEQ ID NO: 17)

5′-cat ggc act cag ctt ctt ctc cag cac cag-3′.

And the following primers were used to make HDAC deficient HDAC7 (H669F/H670F/H709F/H710F, MutHDAC7):
1) H669F/H670F with

(SEQ ID NO: 18)

5′-gtg gtg cgg ccc cca gga ttc ttt gca gat cat tca
aca gc-3′
and

(SEQ ID NO: 19)

5′-gct gtt gaa tga tct gca aag aat cct ggg ggc cgc
acc ac;

2) then use the H669F/H670F mutated construct as template to further mutate H709F/H710F with

(SEQ ID NO: 20)

5′-gta gac tgg gac gtg ttc ttt ggc aac ggc acc cag
c-3′
and

(SEQ ID NO: 21)

5′-gct ggg tgc cgt tgc caa aga aca cgt ccc agt cta
c-3′.

All the mutants were made with QuickChange™ site-directed mutagenesis kit (Stratagene) according to the manufacturer's standard procedure, and confirmed by DNA sequencing.

### ShRNA Vectors and Reagent

TRC shRNAs (Lenti) targeting human TIP60, TRCN0000020315 (sh15), and the Arrest-In transfection reagent (cat no. ATR1741) were purchased from Open Biosystem. The non-target shRNA control vector was purchased from Sigma (cat no. SHC002).

### Cells and Transfections

Human HEK 293T cells were grown in Dulbecco's modified Eagle's medium (DMEM) supplemented with 10% fetal calf serum, penicillin/streptomycin and glutamine. The cells were transfected with 2 μg of each plasmid using Fugene 6 transfect reagent (Roche) according to the manufacturer's instruction manual. Forty-eight hours post transfection cells were used in the indicated assay.

### Cell Lysis, Immunoprecipitation and Immunoblotting

Cell lysates were obtained by cell lysis in RIPA buffer (50 mM Tris-HCl pH 7.4, 0.5% NP-40, 0.25% Na-deoxycholate, 150 mMNaCl, 1 mM EDTA, with 1 mM PMSF, 1 μg/ml each of Aprotinin, leupeptin and pepstatin, 1 mM Na₃VO₄, and 1 mM NaF), followed by immunoprecipitation with indicated antibodies, SDS-PAGE, and analyzed by Western blotting with standard procedures. ECL or ECLP^{plus} Western blotting detection reagents were used (Amersham Biosciences).

### Dual Luciferase Assay

Jurkat transfections and all luciferase assays were performed as follows. Briefly, Jurkat E6.1 T cells were harvested at ˜0.4×10⁶cells/ml growing in antibiotic-free RPMI plus 10% FCS medium, and resuspended at 20×10⁶ cells/ml. A Total of 0.5 ml cells and 20 μg FOXP3 expression vectors or the control empty vector, 15 μg IL-2-Luciferase and 21 μg of TK-Renilla luciferase vectors, as indicated, were electroporated in 0.4 cm gap cuvette (Invitrogen) at LV (low voltage), 310 Volt, 10 ms, using a BTX ECM 2001 electroporation system (Harvard Apparatus, Mass.). 20-24 hours after the electroporation, cells were stimulated with 50 ng/ml of PMA and 1 μM ionomycin for 6-7 hours before lysing cells and analyzed by means of dual luciferase assay normalized with Renilla luciferase activity according to the manufacturer's protocol (Promega). Results presented are the mean of three separate experiments, and the error bars indicate standard deviations.

### Interleukin-2 ELISA

24 hours after transiently transfecting FLAG-tagged FOXP3a or empty control vector, Jurkat T cells were treated with 400 nM Trichostatin A for 4 hours followed by 3 washes in RPMI, then stimulated with plate-bound anti-TCR v beta 8.1 monoclonal antibody plus 1 μg/ml soluble anti-CD28 antibody (PharMingen) for 16 hours. Interleukin-2 secretion was determined by the Human IL-2 (Interleukin-2) ELISA Ready-SET-Go! Kit according to the standard protocol provided by the manufacturer (eBioscience).

### Example 5

Anti-CD3/CD28 stimulation in combination with a high dose of IL-2 was used to expand human Treg cells in vitro as follows. Human FOXP3+CD4+CD25+ T cells were obtained by in vitro expansion as follows. Two hundred million PBLs were stained for CD4 and CD25, and using a Mo Flo high speed sorter, the brightest (top 1%) CD4+CD25+ cells were purified. These cells were stimulated with anti-CD3, anti-CD28 coated beads using a 3 bead to 1 cell ratio or using a cell based αAPC expressing CD64 and CD86 loading with anti-CD3 Ab in the presence of high levels of IL-2 (3000 U/ml) and cultured in RPM! with 10% FCS for the next 20-25 days.

### Example 6

Experiments were done to test the effect of valproic acid (VPA) on autoimmune disease models. Three models were used: a collagen induced arthritis (CIA) model; a non-obese diabetic mouse model; and an experimental autoimmune encephalomyelitis model. Collagen used in the CIA model was collagen from chicken sternal cartilage powder, Type II, from Sigma Cat#C9301. The peptide that was used in EAE experiments was Mouse MOG38-50 peptide (GWYRSPFSRVVHL-SEQ ID NO:1), which was synthesized using>F-moc solid phase methods and purified through HPLC by Invitrogen Life Technologies. For each model tested 7-10 mice were used per group. Results obtained with VPA were compared to those using the negative control phosphate buffered saline. The results are shown below in Table 1.

TABLE 1
Effect of Valproic Acid on Autoimmune Disease Models.

Incidence of
Disease

| Model | PBS | VPA |
|---|---|---|
| Collagen Induced Arthritis | 100% | 29% |
| Non-obese diabetic mouse | 50% | 20% |
| Experimental Autoimmune | 100% | 57%* |
| Encephalomyelitis | | |

1. (canceled)

2. A method for treating an individual who has an autoimmune disorder or treating an individual who has an coronary artery disease or reducing the symptom of allergy of an individual, or treating an individual who has or is at an elevated risk of getting sepsis/toxic shock, or reducing the risk of rejection of an allograft in an individual undergoing immunosuppression, the method comprising the step of administering to an individual a therapeutically or prophylactically effective amount of one or more regulatory compositions to activate the T suppressor cells by increasing the acetylation level and/or protein level of FOXP3.

3. A method for treating an individual who has an autoimmune disorder or treating an individual who has an coronary artery disease or reducing the symptom of allergy of an individual, or treating an individual who has or is at an elevated risk of getting sepsis/toxic shock, or reducing the risk of rejection of an allograft in an individual undergoing immunosuppression. the method comprising:
a) removing peripheral blood mononuclear cells from said individual;
b) treating said peripheral blood mononuclear cells with one or more regulatory compositions to activate T suppressor cells by increasing the acetylation level and/or protein level of FOXP3; and
c) reintroducing treated peripheral blood mononuclear cells to the individual to suppress an aberrant immune response.

4. A method for treating an individual who has an autoimmune disorder or treating an individual who has an coronary artery disease or reducing the symptom of allergy of an individual, or treating an individual who has or is at an elevated risk of getting sepsis/toxic shock, or reducing the risk of rejection of an allograft in an individual undergoing immunosuppression, the method comprising:
a) removing peripheral blood mononuclear cells from said individual;
b) isolating T suppressor cells from other PB MC;
c) treating said T suppressor cells with one or more regulatory compositions to
activate T suppressor cells by increasing the acetylation level and/or protein level of FOXP3; and
d) reintroducing treated T suppressor cells to the individual to suppress an aberrant immune response.

5. A method of any of claims 2-4 further comprising administering to the individual or cell, one or more immunosuppressants that do not activate the T suppressor cells by increasing the acetylation level and/or protein level of FOXP3.

6. The method of claim 5 wherein the one or more immunosuppressants that do not activate the T suppressor cells by increasing the acetylation level and/or protein level of FOXP3 is selected from the group consisting of: corticosteroids, rapamycin, Azathioprine (Imuran), Mycophenolate (MFM or CellCept), Cyclosporine (Sandimmune), Mercaptopurine (6-MP), basiliximab, daclizumab, sirolimus, tacrolimus, Muromonab-CD3, cyclophosphamide, and methotrexate.

7. The method of any of claims 2 to 4 wherein said regulatory composition comprises one or more of deacetylase inhibitors.

8. The method of claim 7 wherein said deacetylase inhibitor is selected from the group consisting of trichostatin A, trapoxin B, butyrates (e.g., sodium butyrate, sodium phenylbutyrate, arginine butyrate, and butyric acid), MS 275-27, m-carboxycinnamic acid bis-hydroxamide, depudecin, oxamflatin, apicidin, suberoylanilide hydroxamic acid, Scriptaid, pyroxamide, valproic acid, 2-amino-8-oxo-9,10-epoxy-decanoyl, 3-(4-aroyl-1H-pyrrol-2-yl)-N-hydroxy-2- propenamide, CI994, Pivanex, FK228, NVP-LAQ824, NVP-LBH589, MS-275, PXDIOI, FR901228.

9. The method of any of claims 2 to 4 wherein said regulatory composition comprises one or more of acetyl transferases enhancer.

10. The method of any of claims 2 to 4 wherein said regulatory composition comprises one or more of reagents that change the expression level or activity of TIP60.

11. The method of any of claims 2 to 4 wherein said regulatory composition comprises one or more of reagents that change the expression level or activity of HDAC7.

12. (canceled)

13. The method of any of claims 2 to 4 wherein said individual has an autoimmune disorder that is caused by a lack of functional suppressor T cells and is selected from the group consisting of multiple sclerosis, diabetes mellitus, rheumatoid arthritis, lupus, Crohn's disease, Hashimoto's disease, polymyositis, inflammatory bowel disease, scleroderma, oophoritis, thyroiditis, Grave's disease, dermatomyositis, pemphigus vulgaris, myasthenia gravis, hemolytic anemia, and Sjogren's disease.

14. The method of any of claims 2 to 4 wherein said individual has coronary artery disease that is caused by atherosclerosis, in which inflammation leads to lesions in the arterial tree.

15. A method for treating cancer, infectious diseases and immune deficiency in an individual by administering to the individual a therapeutically or prophylactically effective amount of one or more regulatory compositions to inactivate the T suppressor cells by reducing the acetylation level and/or protein level of FOXP3.

16-20. (canceled)

21. The method of any of claims 15 16 claim 15 wherein said regulatory composition increases expression level or activity of HDAC7.

22. The method of claim 15 wherein said regulatory composition reduces expression level or activity of TIP60.

23-24. (canceled)

25. A method for vaccinating an individual comprising the step of administering to an individual a vaccine composition in combination with a compound that reduces the acetylation level and/or protein level of FOXP3.

26. The method of claim 25 wherein compound that reduces the acetylation level and/or protein level of FOXP3 inhibits HAT activity or expression.

27. The method of claim 25 wherein compound that inhibits HAT activity or expression is a compound that inhibits TIP60 activity or expression.

28. (canceled)

29. The method of claim 25 wherein said vaccine induces an immune response against one or more pathogen antigens or one or more antigens associated with cancer cells.

30. A pharmaceutical composition comprising a) one or more compounds selected from the group consisting of deacetylase inhibitors, acetyl transferase enhancers, and compounds that inhibit expression of a deacetylase in combination with b) an antigenic polypeptide and/or a nucleic acid molecule that encodes an antigenic polypeptide and/or c) one or more immunosuppressants that do not activate the T suppressor cells by increasing the acetylation level and/or protein level of FOXP3.

31-32. (canceled)

33. The composition of claim 30 comprising one or more immunosuppressants that do not activate the T suppressor cells by increasing the acetylation level and/or protein level of FOXP3 selected from the group consisting of corticosteroids, rapamycin, Azathioprine (Imuran), Mycophenolate (MFM or CellCept), Cyclosporine (Sandimmune), Mercaptopurine (6- MP), basiliximab, daclizumab, sirolimus, tacrolimus, Muromonab-CD3, cyclophosphamide, and methotrexate.

34. A pharmaceutical composition comprising a) one or more compounds selected from the group consisting of acetyl transferase inhibitors, deacetylase enhancers, and compounds that inhibit expression of an acetyl transferase, in combination with b) an antigenic polypeptide and/or a nucleic acid molecule that encodes an antigenic polypeptide.

35. The composition of claim 34 wherein compounds that inhibit expression of acetyl transferase are selected from the group consisting of: 6-(1,3-DiOXo-1H,3H-benzo[de]isoquinolin-2-yl)-hexanoic acid hydroxyamide (scriptaid), garcinol, isothiazolones, Lys-CoA, bromoacetylthio, and Curcumin.

36. (canceled)

37. The composition of claim 34 comprising a vaccine agent selected from the group consisting of a DNA vaccine, a recombinant vector vaccine, a killed or attenuated vaccine, or a subunit vaccine.

38-60. (canceled)
